(19) United States
(12) Patent Application Publication
(10) Pub. No.: US 2004/0033530 A1
(43) Pub. Date: Feb. 19, 2004

(54) High throughput purification, characterization and identification of recombinant proteins

(76) Inventors: Donald E. Awrey, Mississauga, CA (US); Daniel Mamelak, Toronto (CA); Aled Edwards, Toronto (CA); Akil I. Dharamsi, Richmond Hill (CA)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/409,620

(22) Filed: Apr. 8, 2003

Related U.S. Application Data
(60) Provisional application No. 60/370,667, filed on Apr. 8, 2002.

(51) Int. Cl. G01N 33/53; C12Q 1/37

(52) U.S. Cl. 435/7.1; 435/23

(57) ABSTRACT

The invention provides high throughput assays for rapidly and simultaneously purifying, quantifying, determining the solubility profile, determining the purity and identifying a plurality of recombinant proteins. The method comprises affinity protein purification; proteolytic digestion and analysis of the protein fragments by mass spectrometry in multi-well plates.

### RELATED APPLICATION INFORMATION

This application claims the benefit of priority to Provisional Patent Application No. 60/370,667, filed Apr. 8, 2002, which application is hereby incorporated by reference in its entirety.

### BACKGROUND OF THE INVENTION

Although attempts to evaluate gene activity and to decipher biological processes including those of disease processes and drug effects have traditionally focused on genomics, proteomics offers a more direct and promising look at the biological functions of a cell. Proteomics involves the qualitative and quantitative measurement of gene activity by detecting and quantitating expression at the protein level, rather than at the messenger RNA level. Proteomics also involves the study of non-genome encoded events including the post-translational modification of proteins, interactions between proteins, and the location of proteins within the cell. The structure, function, or level of activity of the proteins expressed by a cell are also of interest. Essentially, proteomics involves the study of part or all of the status of the total protein contained within or secreted by a cell.

In order to characterize proteins and design drugs affecting specific proteins, proteins must be available in a significant amount and in a sufficiently pure state. For example, for analyzing a protein by X-ray crystallography, a protein must be soluble and very pure. However, obtaining proteins in large amounts and sufficiently pure state is often impossible, due to, e.g., the lack of expression of certain proteins in well known expression systems or their expression at very low levels; the lack of solubility of certain proteins; and the inability to obtain certain proteins in pure form. At least some of these problems can be resolved by modifying the proteins or by changing the method of their production. Accordingly, it is highly desirable to have a quick and reliable high throughput assay for purifying, determining the solubility profile, the quantity, and the identity of large numbers of recombinant proteins.

### SUMMARY OF THE INVENTION

The invention provides methods for high throughput determination of the identity, quantity and solubility profile of a plurality of recombinant proteins. In one embodiment, the invention comprises (i) providing a plurality of lysates, wherein each lysate comprises a recombinant protein fused to a tag peptide and a proteolytic enzyme recognition site located between the recombinant protein and the tag peptide, wherein the tag peptide and the proteolytic enzyme recognition site are the same for each of the recombinant proteins and wherein each lysate is provided in a well of a multi-well plate; (ii) separating the soluble and the insoluble biological material of the lysates, to obtain from each lysate a fraction comprising the insoluble biological material and a fraction comprising the soluble biological material; (iii) subjecting one or both of the fractions comprising the soluble and insoluble biological material separately to affinity tag protein chromatography in a multi-well plate to obtain affinity purified recombinant proteins from one or both of the fractions of each lysate; (iv) proteolytically digesting the affinity purified recombinant proteins from one or both of the fractions with a proteolytic enzyme in the presence of an internal quantification standard in a multi-well plate, wherein the proteolytic enzyme cleaves the proteolytic enzyme recognition site and wherein the internal quantification standard consists essentially of a chemically modified form of the tag peptide; (v) subjecting the proteolytic fragments to MALDI-TOF, ion trap or electrospray mass spectrometry in a multi-well plate to obtain a mass spectrum; and (vi) determining the identity and quantity of the plurality of recombinant proteins in one or both of the soluble and insoluble fractions, by comparing the intensity of the peak of the tag peptide in the mass spectrum of the soluble or insoluble fraction to that of the internal quantification standard in the mass spectrum of the soluble or insoluble fraction, respectively, to thereby determine the solubility profile and quantity of the recombinant protein.

Determining the solubility profile and quantity of the plurality of recombinant proteins may be conducted using software, e.g., MSQuant. The method may further comprise determining the identity of the plurality of proteins, by comparing the mass spectrum observed with that of proteins in a database, e.g., by software that performs correlative database searching of proteolytic peptide masses from the mass spectrum with that of protein sequences in a database.

In certain embodiments, each lysate is a lysate of a clone of host cells, wherein each clone comprises a recombinant protein linked to a tag peptide and a proteolytic enzyme recognition site located between the recombinant protein and the tag peptide. In other embodiments, the method comprises first providing a plurality of clones of host cells, wherein each clone is provided in a well of a multi-well plate; and lysing the plurality of clones of host cells in the multi-well plate to obtain a plurality of lysates. The host cells may be of prokaryotic or eukaryotic origin. Alternatively, a lysate may derive from an in vitro transcription and translation assay.

The method may further comprise (i) providing a plurality of RNAs encoding the plurality of recombinant proteins, wherein each RNA is provided in a well of a multi-well plate; and (ii) in vitro translating the RNAs to produce a plurality of lysates, wherein each lysate comprises a recombinant protein. Moreover, the method may also comprise providing a plurality of nucleic acids encoding the plurality of recombinant proteins, wherein each nucleic acid is provided in a well of a multi-well plate; and in vitro transcription of the nucleic acids will produce the plurality of RNAs encoding the plurality of recombinant proteins. The method may further comprise amplifying the plurality of nucleic acids in the multi-well plate to obtain amplified nucleic acids prior to in vitro transcription. In another embodiment, the method further comprises isolating the amplified nucleic acids prior to in vitro translation.

The methods may be conducted in multi-well plates, e.g., in a 96-well plate or a 384-well plate. The method may analyze in parallel at least 10, 50, 96, 100, 200, 300, 384, 1000 or more recombinant proteins.

The affinity chromatography may be a chromatography process using a resin selected from the group consisting of a metal ion chelate resin; glutathione-S-transferase (GST) resin; maltose resin; lectin resin; or a resin coupled to a ligand of the tag peptide. In a particular embodiment, the affinity resin is a metal ion chelate resin charged with Ni⁺⁺ resin and the tag peptide contains polyhistidine.

In some embodiments, the proteolytic enzyme is trypsin. In some embodiments, the internal quantification standard is an isotopically labeled form of the tag peptide. The internal quantification standard may be, e.g., ¹⁵N labeled polyhistidine tag. The method may further comprise purifying the proteolytic fragments prior to mass spectrometry, e.g., by chromatography over C₁₈ reverse phase resin.

In another embodiment, the invention provides a method for high throughput determination of the solubility profile and quantity of a plurality of recombinant proteins. The method may comprise one or more of the following steps: (i) providing a plurality of clones of host cells, wherein each clone comprises a recombinant protein linked to a tag and a proteolytic enzyme recognition site located between the recombinant protein and the tag peptide, wherein the tag and the proteolytic enzyme recognition site are the same for each of the recombinant proteins and wherein each clone is provided in a well of a multi-well plate; (ii) lysing the plurality of clones of host cells in the multi-well plate to obtain first lysates; (iii) subjecting the first lysates to centrifugation in a multi-well plate to collect insoluble material in pellets and soluble material in first supernatants; (iv) transferring the first supernatants to wells of a multi-well plate; (v) adding denaturing buffer to the pellets in the multi-well plate to obtain second lysates; (vi) subjecting the second lysates to centrifugation to collect denatured insoluble material in pellets and denatured soluble material in second supernatants; (vii) subjecting one or both of the first and second supernatants separately to affinity tag chromatography in a multi-well plate to obtain one or both of affinity purified soluble and/or denatured soluble recombinant protein fractions; (viii) proteolytically digesting the affinity purified recombinant proteins with a proteolytic enzyme in the presence of an internal quantification standard in a multi-well plate to obtain proteolytic fragments of recombinant proteins, wherein the proteolytic enzyme cleaves the proteolytic enzyme recognition site and wherein the internal quantification standard consists essentially of a chemically modified form of the tag peptide; (ix) purifying the proteolytic fragments in a multi-well plate to obtain purified proteolytic fragments; (x) subjecting the purified proteolytic fragments to MALDI-TOF, ion trap or electrospray mass spectrometry in a multi-well plate; (xi) determining the quantity of the plurality of recombinant proteins in one or both of the soluble and denatured soluble recombinant protein fractions by comparing the intensity of the peak of the tag peptide in the mass spectrum of the soluble and/or denatured soluble recombinant protein fractions to that of the internal quantification standard present in the mass spectrum of the soluble and/or denatured soluble recombinant protein fractions, respectively, to thereby determine the solubility profile and quantity of the recombinant protein; and (xii) using the data from (x) to determine the identity of the recombinant proteins and compare the observed identities with the expected identities as a quality control process.

In yet another embodiment, the invention provides a method for high throughput determination of the quantity of a plurality of recombinant proteins. The method may comprise (i) providing a plurality of purified recombinant proteins, wherein each recombinant protein comprises a tag peptide and a proteolytic enzyme recognition site located between the recombinant protein and the tag peptide, wherein the tag peptide and the proteolytic enzyme recognition site are the same for each of the recombinant proteins and wherein each recombinant protein is provided in a well of a multi-well plate; (ii) proteolytically digesting the recombinant proteins with a proteolytic enzyme in the presence of an internal quantification standard in a multi-well plate, wherein the proteolytic enzyme cleaves the proteolytic enzyme recognition site and wherein the internal quantification standard consists essentially of a chemically modified form of the tag peptide; (iii) subjecting the proteolytic fragments to MALDI-TOF, ion trap or electrospray mass spectrometry in a multi-well plate to obtain a mass spectrum; and (iv) determining the quantity of the plurality of recombinant proteins, by comparing the intensity of the peak of the tag peptide in the mass spectrum to that of the internal quantification standard in the mass spectrum, to thereby determine the quantity of the recombinant protein.

Also within the scope of the invention are kits, e.g., for high throughput purification, determination of the solubility profile and quantification of a plurality of recombinant proteins. A kit may comprise a vector for expressing recombinant proteins in host cells; affinity chromatography resin; a proteolytic enzyme; an internal quantification standard; a matrix for MALDI-TOF mass spectrometry; and instructions for use. A kit may further comprise at least one buffer selected from the group consisting of a lysis buffer; a denaturing buffer; an affinity chromatography binding buffer; an affinity chromatography washing buffer; an affinity chromatography elution buffer; a proteolytic digestion buffer and at least one multi-well plate.

In another embodiment, the invention provides a computer for determining the quantity of a plurality of proteins; identifying a plurality of proteins; and/or determining the solubility profile of a plurality of proteins. A computer may comprise: (a) a machine-readable data storage medium comprising a data storage material encoded with machine-readable data, wherein said data comprises data obtained from MS analysis of a plurality of recombinant proteins according to the method of claim 1; (b) a working memory for storing instructions for processing said machine-readable data of (a); (c) a central-processing unit coupled to said working memory and to said machine-readable data storage medium for extracting information from the data on the machine-readable storage medium; and (d) a display coupled to said central-processing unit for displaying said results.

In yet another embodiment, the invention provides a business method for providing the quantity of a plurality of proteins; identifying a plurality of proteins; and/or determining the solubility profile of a plurality of proteins, comprising, e.g., (a) receiving MS results obtained essentially according to the method of claim 1 from a sender via a network; (b) analyzing the MS results of (a) according to the method of claim 1 to obtain the amount of a plurality of proteins; identifying a plurality of proteins; and/or determining the solubility profile of a plurality of proteins; and (c) sending at least part of the results to the sender via a network.

Advantages of the invention include the ability to rapidly identify and screen large numbers of recombinant proteins for their identity, solubility, and expression profiles, thereby providing a strict level of quality control ensuring that only appropriate clones are selected, e.g., subjected to large scale growth, protein production, biochemical analysis, biophysical analysis, and structural studies using either X-ray crystallography, NMR, or both. Such a level of screening also provides a cost savings advantage since time and money will not be wasted on “dead-end” clones. By the method of the invention, quality is in no way compromised, yet sensitivity is increased. The test expression system is extremely versatile as the quantitative solubility profiles of gene products from both prokaryotic and eukaryotic organisms can be determined.

### DETAILED DESCRIPTION OF THE DRAWINGS

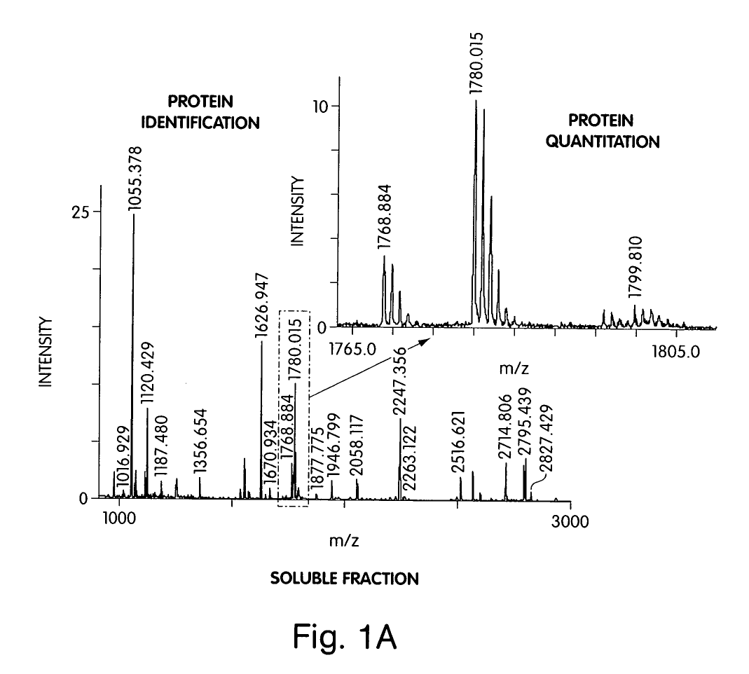

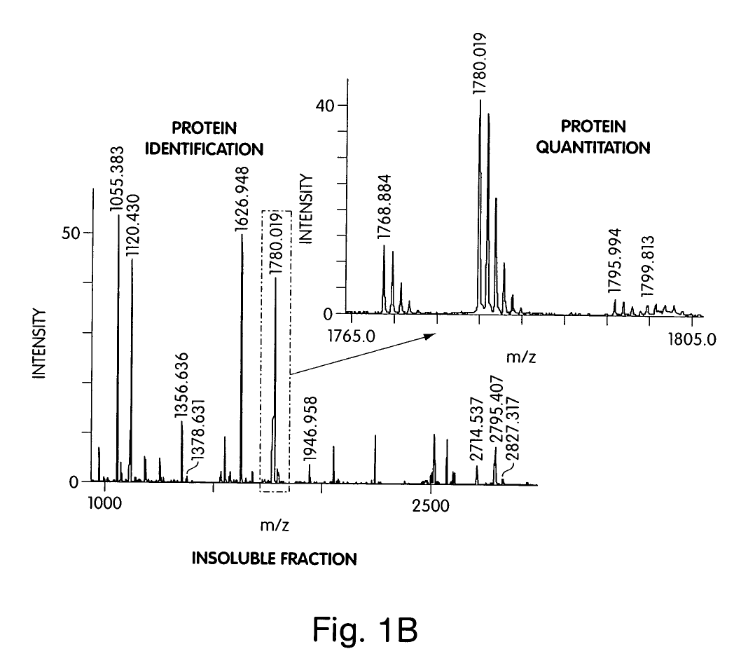

FIGS. 1A and B show exemplary spectra generated by MALDI-TOF MS analysis of a protein purified from the soluble (A) and insoluble (B) fractions. The areas outlined in the spectra are expanded to highlight the location of the ¹⁵N-labeled his tag (m/z=1799) and its non-labeled isoform (m/z=1768) which was released by the recombinant protein upon proteolytic digest.

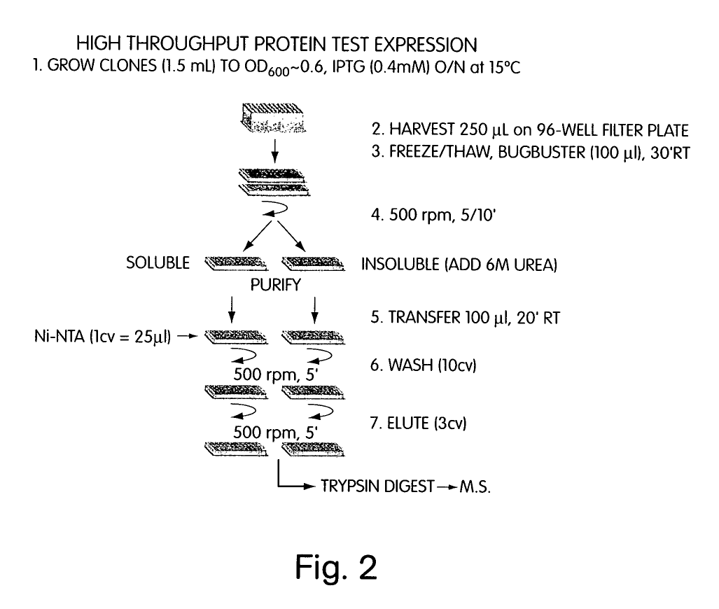

FIG. 2 shows a flow diagram of a 1×96 test expression protocol.

### DETAILED DESCRIPTION OF THE INVENTION

The invention provides a high throughput test expression assay allowing high throughput purification, determination of solubility profiles, quantification, and identification of a plurality of gene products expressed in an expression system. The assay can be performed in a manual, semi-manual or in a fully automated manner. In one embodiment, gene products from 384 clones are analyzed simultaneously. In a preferred embodiment, the assay does not employ gel-based visualization of the purified proteins, e.g., SDS-PAGE combined with Coomassie Blue, or fluorescent based protein staining.

1. Definitions

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

“Solubility profile” of a protein refers to the proportion of protein that is soluble and that which is insoluble in particular conditions.

A “recombinant protein” refers to a protein that is expressed in a prokaryotic or eukaryotic expression system or a protein that is produced in an in vitro transcription and translation system.

“Affinity chromatography” refers to a chromatography process based on the binding of certain molecules to other molecules, e.g., binding of a 6xHis tag to a solid support to which Ni⁺⁺ is attached.

The term “membrane associated protein” refers to a protein that fractionates with the membrane upon lysis of a cell and isolation of the membrane from the cell lysate. Membrane associated proteins include those proteins that are directly associated with the membrane as well as proteins that interact with proteins directly associated with the membrane but are not themselves directly associated with the membrane. In exemplary embodiments, proteins may be associated with either the nuclear membrane, the cellular membrane, or both.

An “internal quantification standard” is a molecule, e.g., a peptide, which, by virtue of its presence in a solution, permits the quantification of other molecules, e.g., peptides, in the solution. An internal quantification standard can be an isotopically labeled peptide tag having the same amino acid sequence as the peptide tag that is linked to a recombinant protein that is being analyzed.

A “multi-well plate” refers to a microtitre plate comprising a plurality of wells. A multi-well plate can have, e.g., 24 wells (e.g., 4×6 array), 96 wells (e.g., 8×12 arrays), 384 wells (e.g., 16×24 array), 864 wells (e.g., 24×36 array), and 1536 wells (e.g., 32×48 array).

“Proteolytic fragments” refers to peptides resulting from the proteolytic, e.g., trypsin, digestion of a protein.

The terms “peptide,” “polypeptide” and “protein” (when a single amino acid chain) are used interchangeably herein.

The term “related polypeptide” or “related protein” refers to the amino acid sequence of a polypeptide that differs from the amino acid sequence of a reference polypeptide by the substitution, addition, and/or deletion of at least one amino acid residue. The term is meant to encompass naturally-occurring proteins, homologs, orthologs, paralogs, fragments, and other equivalents, variants and analogs of the foregoing, and recombinant polypeptides. In an exemplary embodiment, the reference polypeptide is a wild-type polypeptide.

2. Production and Purification of Recombinant Proteins

This section describes exemplary methods for producing proteins in expression systems, recovering the soluble and insoluble fractions, and purifying the protein from the soluble and insoluble fractions. In exemplary embodiments, at least about 0.1 μg, 1 μg, 2 μg, 5 μg, 10 μg, 50 μg, 100 μg, or 1 mg, or more of or purified protein may be obtained from each starting sample (e.g., lysate, etc.) using the methods described herein.

Proteins of the invention can be made in a cell (a “host cell”) or in a lysate, e.g., a lysate prepared from cells. The cells, referred to herein as host cells, can be of prokaryotic or eukaryotic origin. In a preferred embodiment, the nucleic acid encoding a protein of interest is operably linked to one or more transcriptional control sequences, e.g., a promoter and an enhancer. Generally, such nucleic acids are also incorporated into a plasmid or an expression vector, which is then introduced into a host cell to allow expression of the protein. The type of transcriptional control sequences used will depend on the particular expression system used, e.g., whether the system is prokaryotic (e.g., bacterial) or eukaryotic (e.g., yeast, avian, insect or mammalian), or an in vitro transcription system.

In one embodiment, the expression system is a prokaryotic expression system. Generally, a nucleic acid encoding a protein of interest is operably linked to one or more transcriptional control elements, such as a promoter; the nucleic acid is introduced into a prokaryotic host cell; and the host cell is cultured such as to produce the protein of interest. A plasmid for practicing the invention preferably comprises sequences required for appropriate transcription of the nucleic acid in bacteria, e.g., a promoter and a transcription termination signal. The vector can further comprise sequences encoding factors allowing for the selection of bacteria comprising the nucleic acid of interest, e.g., gene encoding a protein providing resistance to an antibiotic and sequences required for the amplification of the nucleic acid, e.g., a bacterial origin of replication. Exemplary vectors for the expression of a protein in prokaryotic cells, such as E. coli, include plasmids of the types: pBR322-derived plasmids, pEMBL-derived plasmids, pEX-derived plasmids, pBTac-derived plasmids and pUC-derived plasmids.

Any of the numerous prokaryotic expression systems known in the art can be used in the invention. Numerous systems are commercially available, e.g., from Novagen and InVitrogen. Exemplary systems are described below. The expression vector can be introduced into the prokaryotic host cells according to methods known in the art, e.g., heat shock transfection of chemically competent cells or electroporation. Host cells having incorporated the expression vector are then identified and used for the production of the protein of interest.

The nucleic acid encoding the protein of interest can be under the control of an inducible promoter. Such promoters are well known in the art and are found in commercially available vectors. The presence of an inducible promoter facilitates expression of proteins that may otherwise be toxic to the host cells. For example, the powerful phage T5 promoter, which is recognized by E. coli RNA polymerase, can be used together with a lac operator repression module to provide tightly regulated, high level expression or recombinant proteins in E. coli. In this sytem, protein expression is blocked in the presence of high levels of lac repressor. Such vectors are available commercially, e.g., from Qiagen (Chatsworth, Calif.; QIAexpress pQE vectors). Other inducible promoters are those that are inducible by iron or in iron-limiting conditions. Examples of iron-regulated promoters of FepA and TonB are known in the art and are described, e.g., in the following references: Headley, V. et al. (1997) Infection & Immunity 65:818; Ochsner, U. A. et al. (1995) Journal of Bacteriology 177:7194; Hunt, M. D. et al. (1994) Journal of Bacteriology 176:3944; Svinarich, D. M. and S. Palchaudhuri. (1992) Journal of Diarrhoeal Diseases Research 10:139; Prince, R. W. et al. (1991) Molecular Microbiology 5:2823; Goldberg, M. B. et al. (1990) Journal of Bacteriology 172:6863; de Lorenzo, V. et al. (1987) Journal of Bacteriology 169:2624; and Hantke, K. (1981) Molecular & General Genetics 182:288.

In another embodiment, an inducible promoter is used which can be activated by temperature, isopropylthio-beta-galactoside (IPTG), NaCl, or other stimuli. Using this inducible system, a protein of interest can be produced, e.g., as follows. Transformed bacteria are grown in liquid media, e.g., LB liquid media, at 37° C. to an optical density of about 0.5 to 0.7, preferably, about 0.6, at 600 nm. At that point, IPTG is added to a final concentration of about 0.1 to 1 mM, preferably from 0.2 to 0.5 mM and even more preferably about 0.4 mM, and the culture is incubated at about 10 to 20° C., preferably about 15° C. for about 10 to about 24 hours, preferably about 12 to about 15 hours. In another embodiment, IPTG is added to a final concentration of about 0.5 mM to 3 mM, preferably about 1 mM, and the culture is incubated at about 37° C. for about 3 to about 5, preferably about 4 additional hours. Induced bacterial cultures expressing recombinant proteins can be harvested by centrifugation or filtration. In another embodiment, a eukaryotic expression system is used. Eukaryotic protein expression systems can be based on virtually any eukaryotic species, e.g., mammalian cells, insect cells, yeast cells and plant cells. Generally, a nucleic acid encoding a protein of interest is operably linked to at least one transcriptional control element, e.g., a promoter and an enhancer. Eukaryotic transcriptional control elements are well known in the art and are described, e.g., in Goeddel; Gene Expression Technology: Methods in Enzymology 185, Academic Press, San Diego, Calif. (1990). Expression systems and appropriate transcriptional control sequences are further described below.

Preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2^{nd} Ed., ed. by Sambrook, Fritsch and Maniatisi (Cold Spring Harbor Laboratory Press: 1989) Chapters 16 and 17.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin, zeomycin, bleomycin, DHFR, or neomycin can be used for selection of prokaryotic or eukaryotic host cells containing the recombinant vector.

The recombinant proteins can also be produced in an in vitro system, e.g., in an in vitro transcription and translation system. Many vectors for in vitro transcription are available commercially. These may contain one or more of the promoters SP6, T3 and T7 and may additionally contain a polyA sequence at the 3′ end of the polylinker in which the DNA of interest is inserted. A “polylinker” refers to a nucleotide sequence containing several restriction enzyme recognition sites. Examples of vectors include the series of SP6 vectors, e.g,. SP64 (Krieg and Melton, infra), BlueScript, and pCS2+. Vectors that can be used for in vitro transcription are also described, e.g., in U.S. Pat. No. 4,766,072. In vitro transcription can be conducted with a nucleic acid that is not per se a vector, but merely contains the elements necessary for in vitro transcription. For example, such a template nucleic acid may comprise an RNA polymerase promoter located upstream of the sequence to transcribe. Such template nucleic acid can be obtained, e.g., by polymerase chain reaction (PCR) amplification of a sequence of interest using a primer that contains an RNA polymerase promoter. PCR amplification methods are well known in the art.

An in vitro transcription reaction can be carried out according to methods well known in the art. Kits for performing in vitro transcription kits are also commercially available from several manufacturers. In an illustrative embodiment, an in vitro transcription reaction is carried out as follows. A vector containing an RNA Polymerase promoter and an insert of interest is preferably first linearized downstream of the insert, by e.g., restriction digest with an appropriate restriction enzyme. The linearized DNA is then incubated for about 1 hour at 37 or 40° C. (depending on the RNA polymerase) in the presence of ribonucleotides, an RNAase inhibitor, an RNA polymerase recognizing the promoter that is operably linked upstream of the insert to be transcribed, and an appropriate buffer containing Tris.Cl, MgCl₂, spermidine and NaCl. Following the transcription reaction, RNAase free DNAse can be added to remove the DNA template and the RNA can be purified by, e.g., a phenol-chlorophorm extraction. Usually about 5-10 μg of RNA can be obtained per microgram of template DNA. Further details regarding this protocol are set forth, e.g., in Molecular Cloning A Laboratory Manual, 2nd Ed., ed. by Sambrook, Fritsch and Maniatis (Cold Spring Harbor Laboratory Press: 1989).

In another embodiment, the RNA is “capped” prior to use with an in vitro translation system. In certain situations, efficient translation of eukaryotic RNA requires that the 5′ end of an RNA molecule is “capped”, i.e., that the 5′ nucleotide at the 5′ end of the RNA has a 5′-5′ linkage with a 7-methylguanylate (“7-methyl G”) residue. The presence of a 7-methyl G on an RNA molecule in a 5′-5′ linkage is referred to as a “cap.” It has been proposed that recognition of the translational start site in mRNA by the eukaryotic ribosomes involves recognition of the cap, followed by binding to specific sequences surrounding the initiation codon on the RNA. Accordingly, it is possible that in certain embodiments of the invention, capping of the RNA synthesized in vitro prior to contacting the RNA with an in vitro translation system improves the translation efficiency of the RNA. Thus, in one embodiment, the RNA is contacted with methyl-7 (5′)PPP(5′)guanylate (available, e.g., from Boehringer Mannheim Biochemicals) in the presence of an in vitro transcription reaction mixture, to obtain capped RNA. In the case of in vitro transcribed RNA, capping is preferably carried out during in vitro transcription, but can also be carried out during in vitro translation by, e.g., addition of a cap analog (GpppG or a methylated derivative thereof). Cap analogs and protocols pertaining to their use are commercially available, e.g, in in vitro transcription and/or translation kits.

In vitro synthesized RNA can be in vitro translated using an in vitro translation system. The term “in vitro translation system”, which is used herein interchangeably with the term “cell-free translation system” refers to a translation system which is a cell-free extract containing at least the minimum elements necessary for translation of an RNA molecule into a protein. An in vitro translation system typically comprises at least ribosomes, tRNAs, initiator methionyl-tRNA^{Met}, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and are commercially available. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes.

An alternative expression system which can be used to express a recombinant protein is an insect system. For example, a baculovirus expression system can be used. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In another insect system, Autographa californica nuclear polyhedrosis virus (AcNPV) is used as a vector to express foreign genes. The virus grows in Spodoptera frugiperda cells. The gene sequence may be cloned into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983, J. Virol., 46:584, Smith, U.S. Pat. No. 4,215,051).

In a specific embodiment of an insect system, the DNA encoding the subject polypeptide is cloned into the pBlueBacIII recombinant transfer vector (Invitrogen, San Diego, Calif.) downstream of the polyhedrin promoter and transfected into Sf9 insect cells (derived from Spodoptera frugiperda ovarian cells, available from Invitrogen, San Diego, Calif.) to generate recombinant virus. After plaque purification of the recombinant virus high-titer viral stocks are prepared that in turn would be used to infect Sf9 or High Five™ (BTI-TN-5B1-4 cells derived from Trichoplusia ni egg cell homogenates; available from Invitrogen, San Diego, Calif.) insect cells, to produce large quantities of appropriately post-translationally modified protein.

In cases in which plant expression vectors are used, the expression of a protein may be driven by any of a number of promoters. For example, viral promoters such as the ³⁵S RNA and 19S RNA promoters of CaMV (Brisson et al., 1984, Nature, 310:511-514), or the coat protein promoter of TMV (Takamatsu et al., 1987, EMBO J., 6:307-311) may be used; alternatively, plant promoters such as the small subunit of RUBISCO (Coruzzi et al., 1994, EMBO J., 3:1671-1680; Broglie et al., 1984, Science, 224:838-843); or heat shock promoters, eg., soybean hsp 17.5-E or hsp 17.3-B (Gurley et al., 1986, Mol. Cell. Biol., 6:559-565) may be used. These constructs can be introduced into plant cells using Ti plasmids, Ri plasmids, plant virus vectors; direct DNA transformation; microinjection, electroporation, etc. For reviews of such techniques see, for example, Weissbach & Weissbach, 1988, Methods for Plant Molecular Biology, Academic Press, New York, Section VIII, pp. 421-463; and Grierson & Corey, 1988, Plant Molecular Biology, 2d Ed., Blackie, London, Ch. 7-9.

Secreted proteins can be collected in the supernatant. In the case of non-secreted proteins, host cells can be lysed after the recombinant protein has been expressed. In a preferred embodiment, essentially all of the host cells are lysed. Preferably, at least about 70%, 80%, 90%, 95%, 99% or more of the host cells are lysed. Bacteria can be lysed, e.g., with the Bugbuster™ Bacteria Lysis Solution (Novagen, Wis.) according to the recommended protocol. Eukaryotic cells can be lysed, e.g., with mild detergents and/or physical cell disruption, e.g., sonication, according to methods known in the art. The lysate is then centrifuged, the soluble proteins are collected in the supernatant and the insoluble proteins are collected in the pellet.

It may be beneficial, at least in certain circumstances to add a reagent that helps in achieving complete lysis and/or reduces the viscosity of the mixture, e.g., by degrading and/or removing nucleic acids from the cell lysate. One such reagent that can be added is Benzonase™ (Merck KgaA, Darmstadt, Germany). Methods for testing lysis of host cells are known in the art and include, e.g., staining the lysate with a dye that identifies whole cells.

Synthesis of recombinant proteins can be adapted to high throughput, e.g., in multi-well plates. For example, proteins can be expressed in multi-well plates using the Rapid Translation System of Roche (RTS 100 E. coli HY Kit; Roche). This kit contains everything needed to perform protein expression in tubes or multi-well plates, and includes E. coli lysate, reaction mix, amino acid mixture (without methionine), methionine, reconstitution buffer, GFP control vector, and 200 μl thin-walled tubes.

In certain high throughput embodiments, nucleic acids encoding proteins of interest for use in an in vitro transcription and translation assay, such as that of the RTS 100 system from Roche, are prepared in a multi-well plate, in which it is then transcribed and translated. For example, a nucleic acid comprising a sequence encoding a protein of interest is incubated in a well of a multi-well plate together with two primers and reagents for conducting PCR. One of the primers can comprise at its end a promoter necessary for in vitro transcription, e.g., an SP6, T3 or T7 bacteriophage promoter. For example, the amplified product can comprise the promoter at its 5′ end, to permit in vitro transcription. After conducting a PCR reaction to amplify the nucleic acid encoding the protein of interest, and optionally linking a promoter to it, the nucleic acid is used in an in vitro transcription reaction. The method may comprise a step of removing certain reagents used in the PCR reaction prior to in vitro transcription and translation. For example, one or both primers can be removed from the reaction. Alternatively, the PCR product can be purified away from some or most of the PCR reagents. For example, the PCR product can be synthesized with a label (which will essentially not affect the transcription of the PCR product), e.g., biotin, and the PCR products isolated on an avidin or streptavidin solid surface, e.g., beads.

In a preferred embodiment of the invention, proteins are expressed as fusion proteins comprising an affinity peptide which is used in affinity purification of the protein. Accordingly, an affinity peptide or “tag peptide” is linked to the carboxy- or amino-terminus of a protein or it can be internal to the protein. A fusion protein can be purifed by using a ligand specific for the tag peptide, that is, e.g., immobilized to a solid surface, e.g., beads. After incubation of a lysate containing a fusion protein with a solid surface containing a ligand to the tag peptide, to allow binding, the solid surface can be washed, and the fusion protein can be eluted from the solid surface. This can be accomplished with the use of an affinity chromatography system. The ligand can be immobilized on planar surfaces, magnetic beads, tubing, microplates, and the like.

It should be recognized that a recombinant protein fused to a tag peptide or other second polypeptide is in a sufficiently purified form to allow MS analysis, since the mass of the tag peptide will be known and can be considered in the determination. The tag peptide can also be cleaved from the polypeptide prior to the MS analysis, as described infra.

The nature of a tag will depend on the particular affinity purification system used. Various systems are available. In one embodiment, the affinity chromatographic system is immobilized metal affinity chromatography (IMAC), which is based on binding of a tag to a metal ion resin. Metal ions can be, e.g., zinc, nickel, or cobalt ions. The tag can be a polyhistidine sequence, which interacts specifically with metal ions such as nickel, cobalt, iron, or zinc. A polyhistidine tag can be 2xHis; 3xHis; 4xHis; 5xHis; 6xHis; 7xHis; 8xHis or other, provided that it binds essentially specifically to a metal ion. The tag can also be a polylysine or polyarginine sequence, comprising at least four lysine or four arginine residues, respectively, which interact specifically with zinc, copper or a zinc finger protein.

Commercially available systems for IMAC include the following systems, which are sold as kits and as individual components, e.g., vectors, bacterial strains, affinity resins and instructions for use: QIAexpress Ni-NTA Protein Purification System of Qiagen (Qiagen, Calif.); HAT™ Protein Expression & Purification System (Clontech, Palo Alta, Calif.); pTrcHis Xpress™ Kit (InVitrogen); and BugBuster™ His•Bind® Purification Kit (Novagen).

Polyhistidine tagged recombinant proteins can be purified on nickel affinity chromatography as follows. Ni-agarose beads are equilibrated by washing twice with a 5 times volume of binding buffer, e.g., 50 mM Hepes pH 7.5, 500 mM NaCl, 5% glycerol, and 5 mM imidazole. The binding buffer can also be 50 mM Tris, pH 7.5, 150 mM NaCl, 2.5 mM MgCl₂, 1 mM thiamin diphosphate (ThDp), 1 mM 2-mercaptoethanol. The binding buffer can also be combinations of the two buffer described, or have yet different ingredients, which a person of skill in the art can readily determine. The supernatant from the centrifuged cell lysate or the pellet is added to the equilibrated Ni-agarose beads. The lysate/Ni-agarose mixture is incubated at room temperature or on ice for approximately 20 minutes, optionally with occasional mixing to keep the beads in suspension. The non-specifically bound proteins can be removed with a wash buffer, which can be the same as the binding buffer with the addition of about 10-70 mM imidazole, preferably about 20-50 mM imidazole. Additionally, the salt concentration can be increased, e.g., from 150 mM NaCl in the binding buffer to 300 mM NaCl in the wash buffer. Bound protein is eluted with elution buffer, which can be identical to the binding buffer with the addition of about 200 mM to about 1 M imidazole, preferably about 300 mM to 700 mM imidazole and most preferably about 500 mM imidazole. If desired, protein concentrations can be estimated by using the Bio-Rad™ protein assay and protein purity can be assessed by SDS-PAGE and Coomassie blue staining. The protein samples may be flash-frozen and stored at −80° C. at this point.

In a preferred embodiment, the invention comprises purifying a plurality of recombinant proteins in multi-well plates. The affinity resins may be present on magnetic beads, thereby allowing easy removal of the beads from the wells.

In a preferred embodiment, which can be a high throughput embodiment, expression and purification is conducted as follows. A bacterial colony is inoculated into 1000 μl growth medium, e.g., LB, and the appropriate antibiotic, e.g., ampicillin at 50 μg/ml, and incubated overnight at 37° C. to obtain an essentially saturated culture. 25 μl of the culture is used to start a culture in 1.5 ml LB containing the appropriate antibiotic, e.g., ampicillin, and the culture is grown to O.D.₆₀₀ of about 0.5-0.7. At that point, the expression of the recombinant protein is stimulated by the addition of 0.4 mM IPTG, followed by overnight culture at about 15° C. The O.D.₆₀₀ is then measured to confirm cell growth and to determine the density of the bacterial cells in the harvested culture media. 250 μl of overnight induced culture is transferred onto Millipore Multiscreen Plates-0.22 μm PVDF membrane (cat.# MAGVN2250), and the plates are centrifuged at about 500 rpm for about 10 minutes. This step can be repeated with the rest of the overnight culture, or with as much of the culture as necessary. The filter plate is then placed at −20° C. to freeze the pellet for at least about 30 minutes. The filter plate is removed from the freezer, thawed, and 100 μl of native binding buffer containing 1×Bugbuster (for Vt=30 ml, 27 ml binding buffer, 3 ml 10×Bugbuster™ (Novagen, Wis.), 300 μl protease inhibitors (50 mM PMSF and 50 mM benzamidine), 30 μl Benzonase™ (Novagen, Wis.)) is added. The native binding buffer can be 50 mM Hepes pH 7.5, 500 mM NaCl, 5% Glycerol, and 5 mM imidazole. The plate is gently shaken at room temperature for about 30 minutes. The plate is then centrifuged at about 500 rpm for about 5 minutes to collect the soluble fraction in the supernatant and the insoluble fraction on the filter membrane.

Solubilization of the insoluble fraction can be conducted by adding denaturing buffer to the pellet containing the insoluble proteins. For example, 100 μl of denaturing binding buffer (same as the binding buffer with the addition of 6 M Urea, 10.8g/30 ml) is added to each well of the filter plate containing the insoluble proteins and cell debris, and the plate is gently shaken for 10 minutes at room temperature. The plate is then centrifuged for about 10 minutes at about 500 rpm to separate the denatured soluble proteins from insoluble proteins and cell debris. This step of adding denaturing binding buffer, shaking the plate and centrifugation can be repeated as necessary, to solubilize more proteins from the insoluble fraction.

100 μl of the soluble fractions or the solubilized insoluble fraction are added to 50 μl of Ni-NTA (50% slurry), pre-equilibrated with soluble binding buffer and aliquoted into Millipore Multiscreen plates (0.221 μm PVDF) and incubated at room temperature for a minimum of 20 minutes to allow the recombinant protein to bind to the resin. The plates are centrifuged for about 5 minutes at about 500 rpm and the resin is recovered in the retentate. The resin is then washed twice by addition of 250 μl of wash buffer (same as binding buffer with the addition of 50 mM imidazole) (Vt=200 ml**) and centrifuged for about 5 minutes at about 500 rpm. This step can be repeated as desired to eliminate non-binding and non-specific binding proteins. 75 μl elution buffer (same as binding buffer with the addition of 500 mM imidazole) (Vt=50 mL**) is added, the plate shaken, centrifuged, and the filtrate is recovered. The filtrate can then be subjected to tryptic digestion, as described below. The volumes of reagents listed as ** are for an automated procedure and could be reduced if performed manually.

In another embodiment, the tag peptide comprises a glutathione-S-transferase (GST) fusion protein and the affinity purification comprises using glutathione, GST or an antibody to GST. Systems for expressing and purifying recombinant proteins comprising a GST tag are available from Novagen as BugBuster™ GST•Bind™ Purification Kit and GST•Tag™ Assay Kit. Exemplary vectors for producing such fusion proteins include the pGEX prokaryotic expression vectors from Pharmacia (Piscataway, N.J), e.g., pGEX-5. GST fusion proteins can be affinity purified using glutathione-Sepharose (Sigma Chem. Co.; St. Louis, Mo.) resin; GST-sepharose (Phamarcia-LKB); resin linked to an antibody specific for GST, e.g., mouse anti-GST-Sepharose® 4B (Zymed Laboratories). Protein purification can be performed as described, e.g., in Kuge et al. (1997) Protein Science 6: 1783.

Other affinity purification systems comprise a T7 tag, e.g., available in the T7•Tag® Purification Kit (Novagen); an S tag or thioredoxin (trxA) tag (Novagen); and a Self-Cleavable Chitin-binding Tag, e.g., in the IMPACT™-TWIN System and IMPACT™-CN System (New England Biolabs); or a myc epitope or a peptide portion of the Haemophilus influenza hemagglutinin protein, against which specific antibodies can be prepared and also are commercially available. Other affinity systems include maltose sepharose or agarose affinity chromatrography using a maltose binding protein, and lectin affinity chromatography.

Additional affinity purification systems are based on the interaction between a tag peptide and an antibody to the tag peptide. Tag specific antibodies can be raised using a protein containing the tag peptide, or a peptide portion thereof, as an immunogen. Such an immunogen can be prepared from natural sources, produced recombinantly, or can be synthesized using routine chemical methods. An otherwise non-immunogenic epitope can be made immunogenic by coupling the hapten to a carrier molecule such bovine serum albumin (BSA) or keyhole limpet hemocyanin (KLH), or by expressing the epitope as a fusion protein. Various other carrier molecules and methods for coupling a hapten to a carrier molecule are well known in the art (see, for example, Harlow and Lane, “Antibodies: A laboratory manual” (Cold Spring Harbor Laboratory Press 1988)).

An anti-tag peptide antibody can be a naturally occurring antibody or a non-naturally occurring antibody, including, for example, a single chain antibody, a chimeric antibody, a bifunctional antibody or a humanized antibody, as well as an antigen-binding fragment of such antibodies. Such non-naturally occurring antibodies can be constructed using solid phase peptide synthesis, can be produced recombinantly or they can be obtained, for example, by screening combinatorial libraries containing of variable heavy chains and variable light chains (see Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Hilyard et al., Protein Engineering: A practical approach (IRL Press 1992); Borrabeck, Antibody Engineering, 2d ed. (Oxford University Press 1995); Harlow and Lane, “Antibodies: A laboratory manual” (Cold Spring Harbor Laboratory Press 1988)).

Methods for raising polyclonal antibodies, for example, in a rabbit, goat, mouse or other mammal, are well known in the art (Harlow and Lane, “Antibodies: A laboratory manual” (Cold Spring Harbor Laboratory Press 1988)). Monoclonal antibodies can be obtained using methods that are well known and routine in the art (Harlow and Lane, “Antibodies: A laboratory manual” (Cold Spring Harbor Laboratory Press 1988)). Essentially, spleen cells from a mouse immunized with a polypeptide of interest, or a peptide portion thereof, can be fused to an appropriate myeloma cell line such as SP/02 myeloma cells to produce hybridoma cells. Cloned hybridoma cell lines can be screened using the immunizing polypeptide to identify clones that secrete appropriately specific antibodies. Hybridomas expressing antibodies having a desirable specificity and affinity can be isolated and utilized as a continuous source of the antibodies. Similarly, a recombinant phage that expresses, for example, a single chain antibody of interest also provides a monoclonal antibody that can used for affinity chromatography.

The ligand to a tag peptide, e.g., an antibody, can be linked to a solid support according to methods known in the art, e.g., using N-hydroxysuccinimide-activated (NHS) activated agarose or spheparose (e.g., Affi-gel (BioRad) and Pharmacia Biotech). N-hydroxysuccinimide-Agarose can also be obtained from Sigma Chemical Co. (St. Louis, Mo.; Cat. # H 3512 or H 8635).

In certain embodiments, it may be desirable to cleave the tag peptide from the recombinant protein. For this purpose, one may insert a proteolytic cleavage site, e.g., an endoprotease cleavage site, between the tag peptide and the recombinant protein, such that after purification, incubation of the protein with the endoprotease results in cleavage of the tag peptide from the recombinant protein. Sequences of proteolytic cleavage sites are well known in the art. Vectors and kits comprising endoprotease cleavage sites located between the tag peptide and the site for insertion of the recombinant protein are available from numerous manufacturers. For example, vectors comprising thrombin or factor Xa cleavage sites are available from Novagen in the S•Tag™ Thrombin Purification Kit; Thrombin Cleavage Capture Kit; and Factor Xa Cleavage Capture Kit. Qiagen sells vectors and a kit for cleavage of the tag using TAGZyme.

A person skilled in the art will recognize that variations can be introduced into the above protocol without significantly changing the result thereof. Furthermore, a person skilled in the art will be able to adapt these protocols for high throughput purification. Parallel purification of numerous samples can be conducted with the help of robots, e.g., QIAGEN BioRobot Systems, which integrate Ni-NTA-based protein purification. This workstation allows automated 96-well purification and assay of 6xHis-tagged proteins using Ni-NTA Magnetic Agarose Beads. The procedure involves lysis of the cells, transfer of samples to a microplate, binding of proteins to Ni-NTA Magnetic Agarose Beads, washing and elution of 6xHis-tagged proteins. It is managed by the QIASoft™ Operating System software from Qiagen. Another workstation that can be used for automated work is Biomek FX Laboratory Workstation (Beckman Coulter; see Examples).

Automated procedures can be followed by using software, e.g., Sample Tracker from Zumatrix (Zumatrix Inc., East Falmouth, Mass.) that track the progress or samples and work through a laboratory. Such programs allow the registration of samples, the creation of worklists, progress/status checking, chain of custody management and reporting. Details of sample submitters, product types and users are all stored within the system.

2. Internal Quantification Standard

In a preferred embodiment, an internal quantification standard is used to quantify the amount of recombinant protein in the sample (“spiking”). In one embodiment, the internal quantification standard is chemically modified, e.g., isotopically-labelled, peptide of known molecular weight to which the relative MS peak intensities of the protein samples are compared. Internal quantification standards can be any protein or peptide of which a chemically modified version can be generated. In a preferred embodiment, the internal quantification standard comprises or has the same amino acid sequence or at least a portion of the amino acid sequence of the tag peptide to which the protein of interest is fused. For example, the internal quantification standard can comprise a labeled form of a polyhistidine tag, GST or maltose binding protein, or portion thereof.

In an even more preferred embodiment, the internal quantification standard comprises one or more isotopes, e.g., ¹⁵N substituted for the normal ¹⁴N. Other isotopes that can be used include carbon-13 (¹³C), deuterium (²H), and oxygen-18 (¹⁸O), or any other isotope of carbon, nitrogen, hydrogen, or oxygen. It is preferable, but not a requirement that the isotope be a stable, non-radioactive isotope of an element naturally occurring in the internal quantification peptide. However, radioactive isotopes, such as hydrogen-3 (³H), carbon-13 (¹³C) and sulfur-35 (³⁵S) can also be used.

In other embodiments, a label is attached to an amino acid of the peptide tag. For example, the nucleophilic thiol group contained in the side chain of reduced cysteine residues can be used for labeling of peptides, as described, e.g., in Griffin and Aebersold (2001) J. Biol. Chem. 276:45497; Kenyon and Bruice (1977) Methods Enzymol. 47: 407 and Sechi and Chait (1998) Anal. Chem. 70:5150.

In another embodiment, the internal quantification standard is a molecule that is closely related to the affinity tag or portion thereof, e.g., a form of the affinity tag or portion thereof that is postranslationally modified or which comprises one or more modified amino acids. The modified form preferably behaves in a similar manner to the non modified form during sample purification and MS analysis, i.e., it is capable of being ionized under similar conditions.

Internal quantification standards can be added at any time of the purification process. It is preferably included in the solutions comprising the recombinant proteins after affinity purification. In an even more preferred embodiment, the internal quantification standard is included in the proteolytic digestion buffer. For example, the internal quantification standard can be added in equal amounts to each of the wells containing a recombinant protein, and optionally other control wells, prior to the proteolytic digestion to ensure a uniform distribution among all digested samples; consistent recoveries following sample cleanup and uniform amounts spotted on MS anchor plates. The standard can be added to the trypsin buffer to ensure equal distribution in each protein sample.

The amount of internal quantification standard added per protein sample can be, e.g., from about 1 μmole to about 1 pmole, preferably around 10 pmole. The internal quantification standard can also serve as a control for the proteolytic digestion. For example, the internal quantification standard can comprise the tag peptide or portion thereof, linked in sequence to a proteolytic enzyme cleavage site and to an unrelated peptide. The internal quantification standard is added to each well prior to the proteolytic digestion. The unrelated peptide is preferably not a peptide that will create background noise during the MS analysis. For example, the unrelated peptide can be a peptide that is known not to be volatized easily or which has a known peak that will not overlap with the peaks of the peptides of the recombinant proteins of interest. Preferably the internal standard would have one, two, three, or four amino acid residues removed by the proteolytic digestion. Thus, the visualization of a peak consisting of the full length internal quantification standard relative to the proteolytic fragment thereof after MS will indicate the efficiency of the protein digestion.

3. Proteolytic Digestion

Purified proteins from both the soluble and insoluble fractions are preferably digested with a proteolytic enzyme, e.g., aminopeptidase M; bromelain; carboxypeptidase A, B and Y; chymopapain; chymotrypsin, clostripain; collagenase; elastase; endoproteinase Arg-C, Glu-C, Asp-N and LysC; Factor Xa; ficin; Gelatinase; kallikrein; metalloendopeptinidase; papain; pepsin; plasmin; plasminogen; peptidase; pronase; proteinase A; proteinase K; subsilisin; thermolysin; thrombin; trypsin, or other suitable proteolytic enzymes prior to MS analysis, such as to produce peptides of a size that can be analyzed by MS. The digest should be essentially complete, e.g., resulting in at least about 70%, preferably at least 80%, 90%, 95% or 99% of the recombinant protein being digested. The proteolytic digests are also referred to as “peptide mixtures.”

In a preferred embodiment, the proteolytic digestion releases the tag peptide from the recombinant protein by cleavage at the proteolytic cleavage site. Thus, the proteolytic digestion can comprise one protease that removes the tag peptide and another protease that cleaves the recombinant protein into peptides of a size appropriate for MS. In certain embodiments, the same proteolytic enzyme removes the tag peptide and cleaves the recombinant protein at several sites.

In one embodiment, 20 μl of protein eluate (supernatant) recovered from the purification assay described in the previous section is added to 80 μl Trypsin Buffer in Nunc 96-well polypropylene plate (cat.# 249946) (for Vt=65 ml, 30.9 ml 100 mM NH₄HCO₃, 30.9 ml H₂O, 3.2 ml 1% CaCl_{2, 2.34} ml of 100 ug/ml trypsin, 202 μl ¹⁵N-His (2007.3 pmoles/50 μl), and incubated overnight at room temperature. The reaction can be stopped with the addition of acetic acid to 1% final concentration.

In certain embodiments, the proteolytic enzyme is attached to a solid support, the lysate containing the protein is incubated with the solid support containing the proteolytic enzyme and the solid support is removed after the proteolytic digestion, as described, e.g., in WO CA99/00640. This allows easy removal of the proteolytic enzyme from the protein fragments prior to MS analysis, and thereby reduces background signals originating from the proteolytic enzyme. Solid supports are well known to those of skill in the art, and include any matrix used as a solid support for linking proteins. Supports, which can have a flat surface or a surface with structures, include, but are not limited to, beads such as silica gel beads, controlled pore glass beads, magnetic beads, Dynabeads, Wang resin; Merrifield resin, sephadex/sepharose beads or cellulose beads; capillaries: flat supports such as glass fiber filters, glass surfaces, metal surfaces (including steel, gold silver, aluminum, silicon and copper), plastic materials (including multiwell plates or membranes (formed, for example, of polyethylene, polypropylene, polyamide, polyvinylidene difluoride), wafers, combs, pins or needles (including arrays of pins suitable for combinatorial synthesis or analysis) or beads in an array of pits; wells, particularly nanoliter wells, in flat surfaces, including wafers such as silicon wafers; and wafers with pits, with or without filter bottoms. A solid support is appropriately functionalized for conjugation of the proteolytic enzyme and can be of any suitable shape appropriate for the support.

A proteolytic enzyme can be conjugated directly to a solid support or can be conjugated indirectly through a functional group present either on the support, or a linker attached to the support, or the proteolytic enzyme or both. For example, a proteolytic enzyme can be immobilized to a solid support due to a hydrophobic, hydrophilic or ionic interaction between the support and the proteolytic enzyme.

A proteolytic enzyme also can be modified to facilitate conjugation to a solid support, for example, by incorporating a chemical or physical moiety at an appropriate position in the polypeptide, generally the C-terminus or N-terminus. It can also be modified at an amino acid in the peptide, for example, to a reactive side chain, or to the peptide backbone. It should be recognized, however, that a naturally occurring amino acid normally present in the proteolytic enzyme also can contain a functional group suitable for conjugating the polypeptide to the solid support. For example, a cysteine residue present in the polypeptide can be used to conjugate the polypeptide to a support containing a sulfhydryl group, for example, a support having cysteine residues attached thereto, through a disulfide linkage.

4. Mass Spectrometric Analysis of Protein Fragments

This section describes the preparation for and MS analysis of the peptide mixtures to obtain an MS spectra that can, e.g., be compared with the spectra of known proteins.

Digested proteins can be desalted and concentrated for increased MS (e.g., MALDI-TOF MS), sensitivity and resolution. The peptide fragments may be purified, for example by use of chromatography. A solid support that differentially binds the peptides and not reagents that were present in the proteolytic digestion may be used. The peptides can be eluted from the solid support into a small volume of a solution that is compatible with mass spectrometry (e.g., 50% acetonitrile/0.1% trifluoroacetic acid). Washing and purification procedures which remove reaction mixture components away from the peptides will increase the resolution of the spectrum resulting from mass spectrometric analysis of the recombinant polypeptide.

In one embodiment, bulk C18 reverse phase resin (Sigma Cat# H-8261) is used, e.g., as follows. Dry resin can be washed with methanol and 75% acetonitrile/1% acetic acid. Resin slurry is then added to proteolytically digested proteins, the mixture is shaken at moderate speed (about 500-700 rpm), e.g., on an orbital shaker, and the supernatant is recovered. Additional resins that can be employed include other silica based resins, styrene resins, and poly(styrene-divinylbenzene) resins or any resin that selectively binds and releases peptides.

MS samples can also be prepared by subjecting the proteolytically digested proteins to ZipTip pipette tips (Millipore), which are pipette tips that contain immobilized C18 attached at their very tip occupying about 0.511 volume. For example, the ZipTips can be wet by aspirating and dispensing 100% methanol 5×; 2% acetonitrile/1% acetic acid (5×); 65% acetonitrile/1% acetic (5×); and 2% acetonitrile/1% acetic acid (5×). The digested proteins are then be bound to the ZipTips, the salts can be removed by washing the ZipTips with 2% acetonitrile/1% acetic acid (5×), and the digested proteins can be eluted by aspirating 65% acetonitrile/1% acetic acid.

In another embodiment using ZipTips, the ZipTips are washed, e.g., with 0.1% trifluroacetic acid (TFA) in acetonitrile, then with 0.1% TFA in 1:1 acetonitrile:water. The ZipTips are equilibrated twice with 0.1% TFA in water. The proteolytically digested protein samples are dissolved in 10 μl of 0.1% TFA, passed through the ZipTips repeatedly by pipeting in and out to bind the sample to the resin. The ZipTips are washed three times with 0.1% TFA, 5% methanol in water, and the samples are eluted from the ZipTips in 1.8 μl of matrix, typically alpha-cyano-4-hydroxycinnamic acid in 0.1% TFA 50% acetonitrile, directly on the MS sample plate.

Multiple samples can be purified simultaneously using, e.g., an electronic pipettor, e.g., the 12-channel Biohit electronic pipettor (Biohit Inc., Neptune, N.J.).

The proteolytically digested proteins (or peptide mixtures) can also be conditioned prior to MS by treating the peptide mixtures with a cation exchange material or an anion exchange material, which can reduce the charge heterogeneity of the peptides, thereby reducing or eliminating peak broadening. In addition, modifying a polypeptide with an alkylating agent such as alkyliodide, iodoacetamide, iodoacetic acid, iodoethanol, or 2,3-epoxy-1-propanol, for example, can prevent the formation of disulfide bonds in the polypeptide, thereby decreasing the complexity of a mass spectrum of the polypeptide. In certain embodiments, disulfide bonds of proteins are reduced, and the free thiols are alkylated after reduction, and preferably prior to digestion of the protein with protease. Reduction can be accomplished by incubation of the protein with a reducing agent, e.g., dithiothreitol. Likewise, charged amino acid side chains can be converted to uncharged derivatives by contacting the polypeptides with trialkylsilyl chlorides, thus reducing charge heterogeneity and increasing resolution of the mass spectrum.

Conditioning also can involve incorporating modified amino acids into the polypeptide, for example, mass modified amino acids, which can increase resolution of a mass spectrum. For example, the incorporation of a mass modified leucine residue in a polypeptide of interest can be useful for increasing the resolution (e.g., by increasing the mass difference) of a leucine residue from an isoleucine residue, thereby facilitating determination of an amino acid sequence of the polypeptide. A modified amino acid also can be an amino acid containing a particular blocking group, such as those groups used in chemical methods of amino acid synthesis. For example, the incorporation of a glutamic acid residue having a blocking group attached to the side chain carboxyl group can mass modify the glutamic acid residue and, provides the additional advantage of removing a charged group from the polypeptide, thereby further decreasing the complexity of a mass spectrum of a polypeptide containing the blocked amino acid. Incorporation of modified amino acids can be done at the time the protein is synthesized. The expression system that lends itself best to including such modified amino acids is an in vitro translation system, as described above.

The peptide mixtures are prepared for MS by mixing the peptide mixtures with a matrix appropriate for the particular MS used. The selection of a solution or reagent system, for example, an organic or inorganic solvent, will depend on the type of mass spectrometry performed, and is well known in the art (see, for example, Vorm et al., Anal. Chem. 66:3281 (1994), for MALDI; Valaskovic et al., Anal. Chem. 67:3802 (1995), for ESI). Mass spectrometry of peptides is also described, for example, in International PCT application No. WO 93/24834 to Chait et al. and U.S. Pat. No. 5,792,664.

A solvent is also selected so as to considerably reduce or fully exclude the risk that the peptides will be decomposed by the energy introduced for the vaporization process. A reduced risk of peptide decomposition can be achieved, for example, by embedding the sample in a matrix, which can be an organic compound such as a sugar, for example, a pentose or hexose, or a polysaccharide such as cellulose. Such compounds are decomposed thermolytically into CO₂ and H₂O such that no residues are formed that can lead to chemical reactions. The matrix also can be an inorganic compound such as nitrate of ammonium, which is decomposed essentially without leaving any residue. Use of these and other solvents is known to those of skill in the art (see, e.g., U.S. Pat. No. 5,062,935).

The peptide mixture and matrix are then applied to a plate for MS analysis, e.g., a metal target plate, according to methods known in the art. In a preferred embodiment, the plates are anchor plates, e.g., plates having a hydrophobic coating and hydrophilic patches (“anchors”). The hydrophobic coating can be, e.g., Teflon. An exemplary plate that can be used is the Bruker Daltonics's Anchor Chip™. Samples can be applied to the plates according to the manufacturer's instructions. Briefly, 1-2 μl sample droplets are deposited onto the plates. The droplets shrink during solvent evaporation and center themselves onto the anchor positions. This allows the peptides to be concentrated in smaller spots and thereby increases the sensitivity of MS detection. Samples can be spotted automatically, e.g., by a modified Gilson 215 (Bruker Daltonics), or a Biomek FX Laboratory Workstation (Beckman Coulter).

The peptide mixtures may also be subjected to a reverse phase column and elution of the peptides from the column directly into a mass spectrometer using an electrospray or nano-electrospray sample introduction interface. For example, peptides may be eluted directly into an ion trap or triple quadrupole mass spectrometer.

Mass spectrometer formats for use in analyzing the peptide mixtures include ionization (I) techniques, such as, but not limited to, matrix assisted laser desorption (MALDI), continuous or pulsed electrospray (ESI) and related methods such as ionspray or thermospray, and massive cluster impact (MCI). Such ion sources can be matched with detection formats, including linear or non-linear reflectron time-of-flight (TOF), single or multiple quadrupole, single or multiple magnetic sector, Fourier transform ion cyclotron resonance (FTICR), ion trap, and combinations thereof such as ESI/time-of-flight. For ionization, numerous matrix/wavelength combinations (MALDI) or solvent combinations (ESI) can be employed. Sub-attomole levels of protein have been detected, for example, using ESI mass spectrometry (Valaskovic, et al., Science 273:1199-1202 (1996)) and MALDI mass spectrometry (Li et al., J. Am. Chem. Soc. 118:1662-1663(1996)).

Accordingly, the following mass spectrometers may be used within the present invention: triple quadrupole mass spectrometers, magnetic sector instruments (magnetic tandem mass spectrometer, JEOL, Peabody, Mass), ionspray mass spectrometers (Bruins et al., Anal Chem. 59:2642-2647, 1987; Fenn et al. J. Phys. Chem. 88:4451-59 (1984); PCT Application No. WO 90/14148; Smith et al., Anal. Chem. 62:882-89 (1990); Ardrey, Electrospray Mass Spectrometry, Spectroscopy Europe 4:10-18 (1992)); electrospray mass spectrometers (Fenn et al., Science 246:64-71, 1989); laser desorption time-of-flight mass spectrometers (Karas and Hillenkamp, Anal. Chem. 60:2299-2301 (1988), and Fourier Transform Ion Cyclotron Resonance Mass Spectrometer (Extrel Corp., Pittsburgh, Mass.). Generally, the method of the invention can be practiced with any mass spectrometer that has the capability of measuring peptide masses with high mass accuracy, precision, and resolution, as well as the capability of measuring the masses of fragments generated from a specific peptide when analyzed under conditions that induce dissociation of the peptide.

Matrix assisted laser desorption (MALDI) is preferred among the mass spectrometric methods herein. Peptide masses are typically accurately measured using a MALDI-TOF or a MALDI-Q-Star mass spectrometer down to the low ppm (parts per million) precision level. MALDI ionization is a technique in which samples of interest, in this case peptides, are co-crystallized with an acidified matrix. The matrix is a small molecule, which absorbs at a specific wavelength, generally in the ultraviolet (UV) range and dissipates the absorbed energy thermally. Typically, a pulse laser beam is used to transfer energy rapidly (e.g., a few ns) to the matrix. This rapid transfer of energy causes the matrix to rapidly dissociate from the surface generating a plume of matrix and the co-crystallized analytes into the gas phase. It is not clear if the analytes acquire their charge during the desorption process or after entering the gas plume of molecules by interacting with the matrix molecules. However, the end result is a small pocket of charged analytes that are present in the gas phase. To date, MALDI has been predominantly coupled in-line with time of flight (TOF) mass spectrometers. The function of a time of flight mass spectrometer is to measure the time that analytes take to travel across a fixed path length (the TOF tube or chamber). The charged analytes present in the plume are therefore transferred to the TOF tube after an appropriate time delay. In order to move the analytes into the TOF tube, a high voltage is applied to the MALDI plate generating a strong electric field between the plate and the entrance of the TOF chamber. Smaller analytes will reach the entrance of the chamber more rapidly than larger analytes (i.e. constant kinetic energy applied, generating different velocity for the analytes). Once in flight, the analytes are in a field-free region and separate along the tube while moving toward the detector. Again, analytes of lesser mass move along the tube faster and reach the detector prior to analytes of greater mass. The detector is in tune with the laser shots and time delay, and measures the peptide and protein ions as they arrive over time. When the mass range is calibrated by using standards of known mass and charge, the time of flight for a given ion can be converted to masses. The end result is a spectrum comparing observed intensity versus mass to charge ratio (m/z). MALDI-TOF mass spectrometry has been described by Hillenkamp et al. (“Matrix Assisted UV-Laser Desorption/Ionization: A New Approach to Mass Spectrometry of Large Biomolecules, Biological Mass Spectrometry” (Burlingame and McCloskey, eds., Elsevier Science Publ. (1990), pp. 49-60).

MALDI-TOF MS is easily performed with modern mass spectrometers. Typically the samples of interest, in this case peptides, are mixed with a matrix mixture and successively spotted onto a polished stainless steel plate (MALDI plate). Commercially available MALDI plates can hold 96, 384, or 1536 samples per plate. The MALDI plate is then installed into the source chamber of a MALDI mass spectrometer. The pulsed laser is activated and the time of flight acquisition triggered. An MS spectrum containing the mass to charge ratios of the peptides is then generated. The charge of molecules ionized by MALDI is typically 1.

Methods for performing MALDI are well known to those of skill in the art. Numerous methods for improving resolution are also known. For example, resolution in MALDI TOF mass spectrometry can be improved by reducing the number of high energy collisions during ion extraction (see, e.g., Juhasz et al. (1996) Analysis. Anal. Chem. 68; 941-946, see also, e.g., U.S. Pat. Nos. 5,777,325, 5,742,049, 5,654,545, 5,641,959, 5,654,545, 5,760,393 and 5,760,393 for descriptions of MALDI and delayed extraction protocols).

MALDI-TOF is useful for high throughput procedures, since it generally takes less than 30 seconds to analyze a sample by MALDI-TOF in an automated procedure, whereas it takes approximately one hour to merely introduce samples into the other kinds of instruments via micro-capillary HPLC. In addition, MALDI-TOF yields a high accuracy peptide mass spectrum (Patterson, Electrophoresis 1995, 16; 1104-14). This sensitive method is able to characterize proteins that are present at very low concentration, as low as sub-picomole levels.

Tandem mass spectrometry or post source decay can be used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al., Rapid Commun. Mass. Spectrom. 1995, 9; 1546-51.

In certain embodiments, e.g., in which it is only desired to obtain quantification and not identification of a protein of interest, the mass spectrometer may be set to monitor only m/z values of ions representative of the molecules of interest so that valuable detection time is not wasted. The form of ionization may also be chosen to favor production of a single type of ion, thus maximizing sensitivity by keeping the ion signal in a single m/z value. This procedure is known as selected ion monitoring (SIM).

5. Data Acquisition and Interpretation

The MS results provide information on the identity of a protein, the amount of protein present in the sample at different times during the purification, its solubility profile and its purity.

The identity of a protein is determined based on the highly accurate determination of the mass of the peptide peaks. The quantity of a protein is based on the comparison between the intensity of the peak of the tag peptide and that of the internal quantification standard. The solubility profile is provided by determining the amount of recombinant protein in the soluble and insoluble fractions and comparing these amounts to each other. The purity of the recombinant protein can be derived from the presence or absence of other peaks in the MS spectrum.

For identifying a protein or confirmation of its identity, the ensemble of the peptide masses observed in a proteolytic digest may be used to search protein/DNA databases in a method often called peptide mass fingerprinting. In this approach protein entries in the databases are ranked according to the number of peptide masses that match to their predicted trypsin digestion pattern. The peptide masses can be searched against in-house proprietary and public databases using a correlative mass matching algorithm. Statistical analysis can be performed upon each protein match to determine the validity of the match. Typical constraints include error tolerances within 0.1 Da for monoisotopic peptide masses. Cysteines are alkylated and searched as carboxyamidomethyl modifications. Identified proteins can be stored automatically in a relational database, e.g., having software links to SDS-PAGE images or ligand sequences. Often, even a partial peptide map of a protein is specific enough for identification of the protein. If no match is found, a more error-tolerant search can be used, for example using fewer peptides or allowing a larger margin for error. In these cases the tentative identity of the interacting protein should be confirmed by a second method.

Commercially available and in-house developed software packages can be utilized to calculate and/or summarize these characteristics/properties in database format. Protein identification and quantification can be obtained within minutes from MALDI-TOF MS generated data that is analyzed by both commercially available and in-house developed software packages.

In a preferred embodiment, the KNEXUS software (Proteometrics LLC, New York, N.Y.) is used. This software interprets and translates the raw mass spectra files and stores the results. Knexus uses the ProFound™ search engine (Proteometrics LLC, New York, N.Y.) for searching protein sequences from database matches, the M/Z (Proteometrics LLC, New York, N.Y.) application to extract peak masses from spectra and the Sonar ms/ms™ (Proteometrics LLC, New York, N.Y.) engine for analyzing information from tandem mass spectrometry. The ProFound™ search engine identifies proteins based on statistics that clearly indicate the probability that a protein identification result is caused by random statistical coincidence. ProFound™ mimics the experiment by calculating the proteolytic peptide masses for all protein sequences in the database and creating a theoretical mass spectrum for each protein sequence. Each theoretical mass spectrum is compared to the experimental mass spectrum, and a score that reflects the similarity is calculated using Bayesian statistics. The algorithm uses detailed information about each individual protein sequence and incorporates additional experimental information (e.g. peptide fragment mass information, amino acid composition or sequence information) when available. Published algorithms provide accurate matches of fragments to proteins, ranking the matches using Bayesian statistics, and a display of errors (so that a requirement for the recallibration of the mass spectrometry spectra may be rapidly diagnosed). Hyperlinks in the Knexus Report connect to database files for the proteins, and connect directly to the Protein Analysis Work Sheet (PAWS; Proteometrics LLC, New York, N.Y.).

The PAWS program was originally designed to manipulate protein sequences and perform calculations that aid in the interpretation of mass spectra. It has the capability of mapping mass spectrum information onto sequences as well as saving complex modifications to proteins. PAWS can read a variety of different protein sequence file formats, including most of the common “flat file” formats. It has been designed to work with the mass spectrum viewing program m/z (Proteometrics LLC, New York, N.Y.), but it is not necessary to use the two programs together.

In another embodiment, MSQuant software package is used. MSQuant is a software package that integrates information generated by Knexus and data provided directly from the MS. The results can be presented in Microsoft Excel files. The results obtained include the following information obtained from Knexus: the clone identification (MS peaks compared to those in the Knexus database); Z % (the likelihood that the protein identified was the protein in the well); the expression (if Z % is <85, then no; if Z % is >/=85, then if the clone identification is the protein found, then yes, otherwise, no); and the molecular weight. The information provided by MSQuant directly from the raw data includes: the % solubility (the ratio of soluble protein to insoluble protein); the soluble quantity in mg of protein expressed per litre of growth media (the expression level of soluble protein); the insoluble quantity in mg of protein expressed per litre of growth media (the expression level of insoluble protein). MSQuant determines these numbers from the intensity of the MS peaks of the soluble and insoluble fractions. In particular, MSQuant quantifies solubility and insolubility according to the following equation for the soluble and insoluble fractions:

value=molecular weight×(sample peak intensity/standard peak intensity)×expression factor, in which the sample and standard (internal quantification standard) peaks are extracted from the MS data and the expression factor is determined based on the volume of culture harvested, the volume of the eluate from the protein purification step, the volume of the protein digested, the amount of peptide standard added to the digest, the volume of the purified peptides, and the amount of sample analyzed by mass spectrometry.

MSQuant or other software quantifying results according to the above equation can be implemented in a variety of ways. For example, the software may be implemented as a “macro” built into a Microsoft Excel spreadsheet that can extract data from specified locations and incorporate these values into the equation for determining the expression profiles of recombinant proteins.

Software for identifying proteins and peptide fragments from tandem mass spectrometry, Quadrapole, QTOF, TOF/TOF, Ion Trap and ESI-Nanospray are also publicly or commercially available, e.g., from Proteometrics (New York, N.Y.). For example, results from tandem mass spectra data can be analyzed with the Sonar ms/ms™ algorithm.

Another alogrithm useful for protein analysis is M/Z (em-over-zee), a freeware program distributed by Proteometrics (New York, N.Y.) for the analysis of protein mass spectra.

Another useful resource for protein analysis is Biopolymer markup language (BIOML) from Proteometrics (New York, N.Y.), which is a browser that allows the full specification of all experimental information known about molecular entities composed of biopolymers, for example, proteins and genes. BIOML provides an extensible framework for the annotation of biopolymers and also provides a common vehicle for exchanging this information between scientists using the World Wide Web.

The invention also provides a computer comprising: (i) a machine-readable data storage material encoded with machine-readable data, (ii) a working memory for storing instructions for processing the machine readable data, (iii) a central processing unit coupled to the working memory and the machine-readable data storage material for processing the machine-readable data into results, and (iv) a display coupled to the central processing unit for displaying the results. For example, the computer can be a computer for (i) determining the amount of one or more proteins; (ii) identifying of one or more proteins; and/or (iii) determining the solubility profile of one or more proteins; wherein said computer comprises: (a) a machine-readable data storage medium comprising a data storage material encoded with machine-readable data, wherein said data comprises data obtained from MS analysis; (b) a working memory for storing instructions for processing said machine-readable data of (a); (c) a central-processing unit coupled to said working memory and to said machine-readable data storage medium for extracting information from the data on the machine-readable storage medium; and (d) a display coupled to said central-processing unit for displaying said results.

Thus the machine-readable data storage medium may comprise a data storage material encoded with machine readable data which can comprise portions and/or all of the results obtained from the MS analysis. A system can include a computer comprising a central processing unit (“CPU”), a working memory which may be random-access memory or “core” memory, mass storage memory (e.g., one or more disk or CD-ROM drives), a display terminal (e.g., a cathode-ray tube), one or more keyboards, one or more input lines and one or more output lines, all of which are interconnected by a conventional bidirectional system bus.

Input hardware, coupled to the computer by input lines, may be implemented in a variety of ways. Machine-readable data may be inputted via the use of one or more modems connected by a telephone line or dedicated data line. Alternatively or additionally, the input hardware may comprise CD-ROM or disk drives. In conjunction with the display terminal, the keyboard may also be used as an input device. Output hardware, coupled to computer by output lines, may similarly be implemented by conventional devices. Output hardware may include a display terminal for displaying the results, e.g., in the form of one or more tables. Output hardware might also include a printer, so that a hard copy output may be produced, or a disk drive, to store system output for later use, see also U.S. Pat. No. 5,978,740, Issued Nov. 2, 1999.

In operation, the CPU (i) coordinates the use of the various input and output devices and; (ii) coordinates data accesses from mass storage and accesses to and from working memory; and (iii) determines the sequence of data processing steps. Any of a number of programs may be used to process the machine-readable data of this invention.

The invention thus further provides a machine-readable storage medium comprising a data storage material encoded with machine readable data which, when using a machine programmed with instructions for using said data, is capable of displaying the results of the MS analysis, e.g., an output from MSQuant.

A system for reading a data storage medium may include a computer comprising a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may comprise CD-ROM drives or disk drives. In conjunction with a display terminal, a keyboard may also be used as an input device.

Output hardware may be coupled to the computer by output lines and may similarly be implemented by conventional devices. In operation, a CPU coordinates the use of the various input and output devices, coordinates data accesses from mass storage devices, accesses to and from working memory, and determines the sequence of data processing steps. A number of programs, e.g., listed above, may be used to process the machine-readable data of this invention.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage data.

In certain embodiments, data is obtained from MS, at least a portion of the data is stored into a machine readable storage medium and sent to another location, e.g., via the a network, e.g., the internet. The data sent can then be analyzed at the other location, and the results can be sent back, e.g., in the form of tables, e.g., Microsoft Excel tables. Accordingly, in certain embodiments, the invention provides a method for conducting business, comprising (i) receiving information, e.g., stored on a machine readable storage medium or transmitted through a network, such as the internet, from a person; (ii) running the data received through data analysis software, e.g., MSQuant; and (iii) sending the results of the analysis back to the person, e.g., via the internet.

In yet other embodiments, the invention provides a machine readable storage medium comprising information sufficient for analyzing MS data to provide the identity, quantity and/or solubility profile of a protein. For example, the invention provides a machine readable storage medium, a computer, or a system, comprising MSQuant software.

6. Uses of the Invention

The invention can be used, e.g., to rapidly determine which recombinant proteins from a large group of recombinant proteins are expressed at high levels, are soluble and can be purified to acceptable levels. The invention can also be used to rapidly confirm the identity or determine the identity of numerous recombinant proteins. These assays can be used, e.g., to identify recombinant proteins that can be produced at high levels and at high purity, such as proteins for use as therapeutics. Indeed, it is well known in the art that certain recombinant proteins cannot be expressed at high levels in certain expression systems, are not soluble or cannot be purified to satisfactory levels, but that even slight modifications to the protein, e.g., an amino acid substitution, can change these characteristics. Thus, the invention provides a quick method for identifying the best candidate protein for a particular purpose, e.g., for use as a therapeutic protein.

The method of the invention can also be used to quickly identify proteins which can be used in certain analytical methods, e.g., in biochemical analysis, biophysical analysis, and structural studies using either X-ray crystallography, NMR, or both. Insoluble proteins present additional challenges to biochemical analysis, biophysical analysis, and structural studies using either X-ray crystallography, NMR, or both and the identification of soluble proteins is preferred. Crystal structures of proteins can be used, e.g., in rational drug design.

In other embodiments, methods for determining the effects of variations in growth conditions on recombinant protein expression are provided. For example, host cells comprising a nucleic acid encoding for at least one recombinant polypeptide can be grown under a variety of different growth conditions. The identity, solubility and/or quantity of recombinant protein obtained from the cells can then be compared to determine how the different growth conditions affect protein expression and/or solubility. Growth conditions that may be varied include, for example, the type of host cell used, type of expression vector used, type of media, temperature, presence or absence of a label, time of culture growth, time of induction of an inducibly expressed protein, etc. In an exemplary embodiment, the methods of the invention may be used to determine optimal growth conditions for the production of one or more polypeptides.

In one embodiment, host cells expressing the same protein are grown under a variety of conditions and the conditions which maximize protein expression and/or solubility are determined. In another embodiment, a plurality of different clones of host cells expressing different recombinant polypeptides are grown under a variety of conditions and the conditions which maximize protein expression and/or solubility for the plurality of proteins as a whole are determined.

In yet another embodiment, methods for determining optimal growth conditions for the production of recombinant proteins comprising a label are provided. Examples of labels that may be incorporated into recombinant proteins include, for example, labels that facilitate detection or structural characterization such as isotopic labels for structural characterization using nuclear magnetic resonance and labels useful for structural characterization using x-ray crystallography.

Exemplary isotopic labels include radioisotopic labels such as, for example, potassium-40 (⁴⁰K), carbon-14 (¹⁴C), tritium (³H), sulphur-35 (³⁵S), phosphorus-32 (³²p), technetium-99m (^{99m}Tc), thallium-201 (²⁰¹ Ti), gallium-67 (⁶⁷Ga), indium-111 (¹¹¹In), iodine-123 (¹²³I), iodine-131 (¹³¹I), yttrium-90 (⁹⁰Y), samarium-153 (¹⁵³ Sm), rhenium-186 (⁸⁶ Re), rhenium-188 (⁸⁸ Re), dysprosium-165 (¹⁶⁵Dy) and holmium-166 (¹⁶⁶Ho). The isotopic label may also be an atom with non zero nuclear spin, including, for example, hydrogen-1 (¹H), hydrogen-2 (²H), hydrogen-3 (³H), phosphorous-31 (³¹p), sodium-23 (²³Na), nitrogen-14 (¹⁴N), nitrogen-15 (¹⁵N), carbon-13 (¹³ C) and fluorine-19 (¹⁹F). In certain embodiments, polypeptides may be uniformly labeled with an isotopic label, for example, wherein at least 50%, 70%, 80%, 90%, 95%, or 98% of the possible labels in the polypeptide are labeled, e.g., wherein at least 50%, 70%, 80%, 90%, 95%, or 98% of the nitrogen atoms in the polypeptide are ¹⁵N, and/or wherein at least 50%, 70%, 80%, 90%, 95%, or 98% of the carbon atoms in the polypeptide are ¹³C, and/or wherein at least 50%, 70%, 80%, 90%, 95%, or 98% of the hydrogen atoms in the polypeptide are 2H. In other embodiments, the isotopic label is located in one or more specific locations within the polypeptide, for example, the label may be specifically incorporated into one or more of the leucine residues of the polypeptide. The invention also encompasses the embodiment wherein a single polypeptide comprises two, three or more different isotopic labels, for example, the polypeptide comprises both ¹⁵N and ¹³C labeling.

Exemplary labels for x-ray crystallography include, for example, heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium. In an exemplary embodiment, the label is seleno-methionine.

A variety of methods are available for preparing a polypeptide with a label, such as a radioisotopic label or heavy atom label. For example, in one such method, an expression vector comprising a nucleic acid encoding a polypeptide is introduced into a host cell, and the host cell is cultured in a cell culture medium in the presence of a source of the label, thereby generating a labeled polypeptide. As indicated above, the extent to which a polypeptide may be labeled may vary.

In one embodiment, host cells expressing the same protein are grown in the presence of a label under a variety of conditions and the efficiency of incorporation of the label into the protein under the variety of conditions is determined. The amount of label incorporated, percent of recombinant proteins labeled, solubility profile, and quantity of the recombinant protein obtained may then be compared to evaluate the growth conditions for affects on one or more of protein expression, solubility, and efficiency of labeling conditions. In another embodiment, a plurality of different clones of host cells expressing different recombinant polypeptides are grown in the presence of a label under a variety of conditions and the conditions which maximize protein expression, solubility, and/or labeling for the plurality of proteins as a whole are determined.

In another embodiment, the methods for evaluating growth conditions for the production of a labeled polypeptide may further comprise determination of the amount of label incorporated into the protein. In an exemplary embodiment, the amount of incorporated label may be determined using mass spectrometry, such as MALDI-TOF, ion trap or electrospray mass spectrometry.

In another exemplary embodiment, the invention provides a method for identifying a variant of a protein that has increased expression and/or solubility as compared to the wild-type polypeptide. For example, a plurality of host cells encoding for a plurality of related polypeptides that differ from each other by at least one amino acid addition, substitution, and/or deletion are provided. The level of protein expression and/or solubility obtained for each of the related polypeptides is then determined and compared to identify variants demonstrating increased protein expression and/or solubility. In an exemplary embodiment, a plurality of fragments of a full-length polypeptide are subjected to the methods of the invention to identify one or more fragments that show increased protein expression and/or solubility as compared to the full length polypeptide.

In certain embodiments, the methods of the invention may utilize an activity assay to monitor the function of a polypeptide, characterize the ability of a molecule to bind to a polypeptide, and/or characterize the ability of a molecule to modify the activity of a polypeptide. Both in vitro and in vivo assays may be used in accordance with the methods of the invention depending on the identity of the polypeptide being investigated. Appropriate activity or functional assays may be readily determined by the skilled artisan based on the disclosure herein.

7. Kits

The invention further provides for commercially available kits, e.g., kits for high throughput purification, determination of the solubility profile and quantification of a plurality of recombinant proteins. Kits may comprise a vector for expressing recombinant proteins in host cells or in vitro transcription/translation systems; affinity chromatography resin; a proteolytic enzyme; an internal quantification standard; a matrix for MALDI-TOF mass spectrometry; and instructions for use. Kits may also comprise at least one buffer selected from the group consisting of a lysis buffer; a denaturing buffer; an affinity chromatography binding buffer; an affinity chromatography washing buffer; an affinity chromatography elution buffer; and a proteolytic digestion buffer. Kits can also comprise vessels, e.g., multi-well plates.

The present invention is further illustrated by the following examples, which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### Quantification and Determination of the Solubility Profile of a 6xHis Tagged Recombinant Protein

This example demonstrates the accuracy of the gel-free test expression system described herein.

A gene encoding an alcohol dehydrogenase protein was expressed in E. coli as a 6xHis fusion protein from a pET15b-derived vector. This vector is a modified version of the commercially available, pET1 Sb (Novagen) in that it has a distinct multiple cloning site. Bacterial colonies expressing this and other genes were selected by picking colonies and inoculating into 75 μL of sterile distilled water. 25 μL of the suspension was used in a quality control polymerase chain reaction (QC-PCR) analysis in which the colonies are screened by PCR to determine if they harbour the recombinant gene (cDNA) of interest. 15 μL of the distilled water bacterial suspension was added to 500 μL of LB growth medium containing 50 μg/mL ampicillin in a Whatman high throughput bacterial growth plate (cat. #7701-5205) and incubated at 37° C., 325 rpm until the culture O.D.₆₀₀ reached 0.5-0.7. Expression of the recombinant protein was stimulated by the addition of 0.4 mM IPTG, followed by overnight culture at 15° C., shaking at 325 rpm. 300 μL of overnight induced culture was transferred onto a Millipore Multiscreen Plate-0.22 μm PVDF membrane (cat.# MAGVN2250), and the plate was centrifuged at 800 rpm for 10 minutes. The filter plate was then placed at −20° C. to freeze the pellet for at least about 30 minutes. The pellet was thawed and 100 μL of “native” binding buffer with 1×Bugbuster™ (Novagen, Wis.; for Vt=30 ml, 27 ml binding buffer, 3 ml 10×Bugbuster, 300 μl Protease Inhibitors, 30 μl Benzonase™; Novagen, Wis.) was added. The binding buffer was 50 mM Hepes, 500 mM NaCl, 5% glycerol, 5 mM imidazole. The plate was gently shaken at room temperature for about 30 minutes. The plate was then centrifuged at 800 rpm for 5 minutes to collect the soluble fraction in the filtrate and the insoluble fraction in the retentate.

100 μl of denaturing binding buffer (same as the binding buffer with the addition of 6 M Urea, 10.8 g/30 ml) was added to the filter plate containing the retentate, and the plate was gently shaken for 10 minutes at room temperature. The plate was then centrifuged for 10 minutes at 800 rpm to collect all solubilized proteins in the filtrate.

100 μl of the soluble and denatured soluble fractions were independently added to 50 μl of Ni-NTA (50% slurry), pre-equilibrated with the appropriate binding buffer, aliquoted into a Millipore Multiscreen™ plate (0.22 um PVDF), and incubated at room temperature for about 20 minutes to allow the recombinant protein bind to the resin. The plate was centrifuged for 5 minutes at 800 rpm. The resin was then washed twice with 250 μl of wash buffer (same as binding buffer with the addition of 50 mM imidazole) (Vt=200 ml) and centrifuged for 5 minutes at 800 rpm. 75 μl elution buffer (same as binding buffer with the addition of 500 mM imidazole) (Vt=50 mL) was added, the plate was shaken, centrifuged, and the filtrate was recovered.

The filtrate containing the soluble and solubilized lysates were then subjected to tryptic digestion by adding 80 μl trypsin digestion buffer in a Nunc 96 well polypropylene plate (cat. # 249946) (for Vt=65 ml, 30.9 ml 100 mM NH₄HCO₃, 30.9 ml. H₂O; 3.2 ml. 1% CaCl₂; 2.34 ml 100 ug/ml trypsin; and 202 μl ¹⁵N-6xHis peptide (2007.3 pmoles/50 μl)). The reaction was incubated overnight at room temperature, and the reaction was stopped with 1% acetic acid.

The ¹⁵N-6xHis peptide was prepared by expressing a soluble recombinant protein in minimal media supplemented with ¹⁵NH₄Cl using techniques described in molecular biology manuals. The protein was purified by metal chelate affinity chromatography and the his tag fusion was removed by thrombin digestion. The protein digest was applied to a second metal affinity chelate column and the his tag was eluted from the column using molecular biology techniques described in most molecular biology manuals. The eluted his tag was purified by high pressure reverse phase chromatography and the peak containing the ¹⁵N his tag was characterized by MALDI-ToF MS and amino acid composition analysis. The fractions containing the his tag peptide were pooled, aliquoted, and stored at −70 degrees Celsius. The tryptic fragments were purified using a ZipTip pipette tip (Millipore, cat.# ZTC18S960). The ZipTips were wet by aspirating (five times) and dispensing 100% methanol using the 12-channel Biohit electronic pipettor. The following solutions were each pipetted five times though the ZipTip: a solution of 2% acetonitrile/1% acetic acid; a solution of 65% acetonitrile/1% acetic; and a solution of 2% acetonitrile/1% acetic acid. The digested proteins were bound to the ZipTip by aspirating and dispensing the sample 20 times. Salts were removed by washing the ZipTip five times with 2% acetonitrile/1% acetic acid. 10 ul of 65% acetonitrile/1% acetic acid was aspirated in the ZipTip, and dispensed into a Nunc 96-well microtitre plate (cat. # 249946).

The sample was mixed with a-cyano-4-hydroxycinnamic acid (Fluka cat# 2488791), spotted automatically by a modified Gilson 215 liquid handler or Biomek FX Laboratory Workstation (Beckman Coulter), subjected to MALDI-TOF MS (Reflex IV, Bruker Daltonics) and interpreted with the MSQuant (Integrative Proteomics Inc.) and Knexus (Proteometrics New York, N.Y.) software.

The Reflex IV (Bruker Daltonics) MALDI-TOF instrument was utilized in positive ion mode with the reflectron voltage at 23.0 kV and the pulsed ion extraction delay set at 400 ns. Spectra were acquired automatically over a mass to charge range of 800-3300, and comprised of 200 summed shots of 50-shot steps. The Knexus batch database search settings were as follows: ion m/z values were all (M+H), recalibration of the spectra was performed utilizing the trypsin autolysis peak at m/z 2163.049 and the internal calibrant of the N-terminal His tag peptide at m/z 1768, mass resolution was 6000 and S/N ratio was 1.7. The spectra generated by MALDI-TOF MS of a proteolytic digest of alcohol dehydrogenase is shown in FIGS. 1A and B. Analysis of the spectra using the Knexus software package (Proteometrics, New York, N.Y.) mapped 57% of the peptide fragments to an E. coli protein having a molecular weight of 36 kDa that is present in both the soluble and insoluble fractions. Concomitant computational analysis of the spectra using the MSQuant software package determined that this protein is expressed in the soluble and insoluble fractions at concentrations of 17 and 35 mg/L, respectively. The ability to accurately determine these values was made possible by incorporating a known concentration of an ¹⁵N labeled (His)₆-tag peptide (“spike”) to the trypsin digestion buffer prior to proteolytic digestion of the unkown sample. The intensity of the internal isotopically labeled (His)₆-tag peptide (m/z=1799) was measured against the non-labeled peptide having an m/z of 1768.

### Example 2

### Semi-Automated Analysis

This example describes a semi-automated high throughput method of quantifying, characterizing and identifying recombinant proteins.

This method employed a Biomek FX Laboratory Workstation (Beckman Coulter). Using this workstation, pipetting, diluting and dispensing operations are performed quickly, easily and automatically. The modular platform allows expansion of system capability to include plate heating and cooling, plate washing, high-density transfers, photometric measurement and high-capacity operation. The entire system is controlled by BioWorks™ software with a graphical interface.

Overnight IPTG induced bacterial cultures are produced as described above. A Millipore Multiscreen Plate-0.22 μm PVDF membrane (cat.# MAGVN2250) (referred to herein as “Millipore filter plate” or “filter plate”) is stacked on a Sarstedt shallow well plate using a rectangular blue adaptor for best fit. 300 μl of the overnight IPTG induced bacterial culture is transferred to corresponding wells of a Millipore filter plate. The pump is connected to the vacuum filtration unit. The vacuum filtration unit is assembled with the Sarstedt (collection) plate positioned on the base of the vacuum manifold and covered by the support grid. The filter plate (without the rectangular adaptor) is placed on the vacuum support grid. The pump and vacuum are plugged in at 600 mbar until all cultures have been harvested. The filter plate is placed at −20° C. to freeze the pellets for 30 min (plates can be stored frozen for up to about 1 week).

Bacterial lysis is conducted as follows. The plate is removed and thawed. The method “A 1×96 Test Expression (Add Bugbuster)” is opened on the Biomek FX Laboratory Workstation (Beckman Coulter). The air is turned on from the blue switch located on the upper right side of the Biomek FX Laboratory Workstation (Beckman Coulter) (The arrow indicates “ON”). The method is started by clicking the green play key on the toolbar. The labware is loaded as depicted in the instrument setup window. The Millipore filter plate containing the harvested bacteria is thawed, stacked on top of an empty Sarstedt plate is placed on the deck in the appropriate position (ALP P6). Native binding buffer containing Bugbusterm (Novagen, Wis.) solution (50 mM Hepes pH 7.5, 500 mM NaCl, 5% glycerol, 5 mM imidazole, 1×Bugbuster™, 1 mM PMSF, 1 mM benzamidine, and 1×Benzonase™ (Novagen, Wis.)) is poured into the reservoir and “OK” is clicked. The system will indicate that the plates will be ready for shaking in 45 seconds. “OK” is clicked to continue. The Biomek FX Laboratory Workstation (Beckman Coulter) will transfer 100 μL of native binding buffer containing Bugbuster™ from the reservoir into the filter plate. Upon completion of the method, the air is turned off from the blue switch (the X indicates “OFF”).

The deck is unloaded, and the filter and collection plates are placed on an orbital shaker, set to 900 rpm, for 30 min. The collection plate is replaced with a fresh one labeled, “soluble lysate.” The filter plate is centrifuged at 800 rpm (max) for 7 min. All liquid from the wells of the Millipore plate must go through and there must be an approximately equal volume in each of the wells of the Sarstedt collection plate. The plate is sealed and placed on ice or 4° C. for further use.

A new Sarstedt collection plate (labeled, “insoluble lysate”) is placed below the Millipore filter. The insoluble lysate is collected as follows. The method “B 1×96 Test Expression (Binding Buffer with Urea)” is opened on the Biomek FX Laboratory Workstation (Beckman Coulter). The air is turned on from the blue switch located on the upper right side of the Biomek FX Laboratory Workstation (Beckman Coulter)(the arrow indicates “ON”). The method is started by clicking the green play key on the toolbar. The labware is loaded as depicted by the instrument setup window. The Denaturing Binding Buffer (50 mM Hepes pH 7.5, 500 mM NaCl, 5% glycerol, 5 mM imidazole, 1×Bugbuster™, 1 mM PMSF, 1 mM benzamidine, 1×Benzonase™ (Novagen, Wis.), and 6 M urea) is poured into the appropriate reservoir. “OK” is clicked. The system will indicate that the plates will be ready for shaking in 45 seconds (“OK” is clicked to continue). The Biomek FX Laboratory Workstation (Beckman Coulter) will transfer 100 μL of Denaturing Binding Buffer from the reservoir to the filter plate. Upon completion of the method, the air is turned off from the blue switch (the X indicates “OFF”). The deck is unloaded, and the filter and collection plates are placed on an orbital shaker, set to 900 rpm, for minimum of 20 min. The plate is then centrifuged at 800 rpm (max) for 20 min. All the liquid in the wells of the Millipore plate should pass through. If necessary, the plate can be centrifuged again. The collection plate is labeled “Insoluble” and sealed.

Ni-NTA is prepared as follows. 7 mL of Ni-NTA is dispensed into two 15 mL Falcon Tubes, Tube 1: Ni-NTA and Tube 2: Ni-NTA (6M urea) and centrifuged at 2000 rpm for 3 min. The supernatant is poured off and replaced with native binding buffer in Tube 1 and with denaturing binding buffer in Tube 2. The resin is resuspended, centrifuged and the supernatant removed. This step is repeated twice more for a total of three washes. After the third wash, 6 mL native binding buffer is added to Tube 1 and 6 mL denaturing binding buffer is added to Tube 2. The resin is resuspended by vigorous vortexing or shaking. 430 μL of Ni-NTA in native binding buffer are dispensed (using P1000) into each well of Row H of a first 96-deepwell plate. 430 μL of Ni-NTA in denaturing binding buffer are dispensed (using P1000) into each well of Row H of a second 96-deepwell plate.

The method “C 1×96 Test Expression (Add Ni-NTA and Lysates)” is opened on the Biomek FX Laboratory Workstation (Beckman Coulter). The air is turned on from the blue switch located on the upper right side of the Biomek FX Laboratory Workstation (Beckman Coulter) (The arrow indicates “ON”). The method is started by clicking the green play key on the toolbar. The following aspiration heights box will appear:

| Deepwell Plate Type | “Deepwell_Aspirate_Height” |
|---|---|
| Nunc Deepwell Plate | 2.1 |
| Life Technologies Deepwell Plate | 1.0 |
| Beckman Deepwell Plate | 1.4 |

Depending on the make of the deep well plate to which the Ni-NTA resin has been dispensed, the appropriate deep well aspirate height is entered and “OK” is clicked. The labware is loaded as depicted by the instrument setup window. The empty Millipore filter plates (Soluble and Insoluble) are stacked on “Nunc Support Plates.” “OK” is clicked. The system will indicate that the plates will be ready for shaking in 5 minutes (“OK” is clicked to continue). The Biomek FX Laboratory Workstation (Beckman Coulter) will first transfer 50 μL of Ni-NTA in native binding buffer and 50 ul of Ni-NTA in denaturing binding buffer from the respective deep well reservoirs to the appropriate filter plates. The Biomek FX Laboratory Workstation (Beckman Coulter) will then transfer 100 μL of both the soluble and insoluble lysates to the respective Millipore plates. Upon completion of the method, the air is turned off from the blue switch (the X indicates “OFF”). The deck is unloaded, and the Millipore plates containing soluble and insoluble lysates in Ni-NTA are stacked on empty Sarstetd collection plates and placed on an orbital shaker for 30 min. The plates are then centrifuged at 800 rpm (max) for 7 min.

The method “D 1×96 Test Expression (Add Wash and Elution Buffers)” is opened on the Biomek FX Laboratory Workstation (Beckman Coulter). The air is turned on from the blue switch located on the upper right side of the Biomek FX Laboratory Workstation (Beckman Coulter) (the arrow indicates “ON”). The method is started by clicking the green play key on the toolbar. The labware is loaded as depicted by the instrument setup window. “OK” is clicked. The system will indicate that the plates will be ready for shaking in 2 minutes (“OK” is clicked to continue). The Biomek FX Laboratory Workstation (Beckman Coulter) will transfer 250 μL of native wash buffer and 250 μL of denaturing wash buffer to the appropriate filter plates. The plates are centrifuged for 7 min. at 800 rpm (max). The Millipore filter plate and the volumes of wash in the collection plate are checked to ensure that all wells have been evenly washed. The filtrate is discarded, the Millipore and Sarstedt plates are restacked, and returned to their original position on the Biomek FX Laboratory Workstation (Beckman Coulter). “OK” is clicked. The Biomek FX Laboratory Workstation (Beckman Coulter) will then transfer another 250 μL of both the native and denaturing wash buffers to the respective Millipore plates. The plates are centrifuged for 7 minutes at 800 rpm (max). The Millipore filter plate and the volumes of wash in the collection plate are checked to ensure that all wells have been evenly washed. The filtrate is discarded, the Millipore plates are stacked on new Sarstedt plates (labeled ‘soluble’ and ‘insoluble’), and they are returned to their original position on the Biomek FX Laboratory Workstation (Beckman Coulter). “OK” is clicked. The Biomek FX Laboratory Workstation (Beckman Coulter) will then transfer 75 μL of the Soluble Elution Buffer and 75 μL pf the Insoluble Elution Buffers to the respective Millipore plates. The plates are centrifuged for 7 min. at 800 rpm (max) and the filtrate containing the eluted proteins are saved. Upon completion of the method, the air is turned off from the blue switch (the X indicates “OFF”). The deck is unloaded, the tips, tip boxes and the soluble and insoluble Millipore Filter plates are discarded. The blue rectangular adaptor rings are not discarded.

Trypsin digestion is carried out as follows. Trypsin digestion buffer is prepared by combining 16.6 ml 100 mM NH₄HCO₃; 16.6 ml H₂O; 1.7 ml 1% CaCl₂; 1.26 ml of 100 ug/ml trypsin; and 0.109 ml ¹⁵N His-Tag (2007.3 pmoles/50 ul) The method “E 1×96 Test Expression (Trypsin Digest)” is opened on the Biomek FX Laboratory Workstation (Beckman Coulter). The air is turned on from the blue switch located on the upper right side of the Biomek FX Laboratory Workstation (Beckman Coulter; the arrow indicates “ON”). The method is started by clicking the green play key on the toolbar. The labware is loaded as depicted by the instrument setup window. “OK” is clicked. The system will indicate that the reaction will be complete in 12 hours and that the protein plates will be ready for storage in 3 minutes (“OK” is clicked to continue). The Biomek FX Laboratory Workstation (Beckman Coulter) will mix 80 μL of trypsin digest buffer with 20 μL of purified protein (soluble and insoluble) in the appropriate digest plates. The Biomek FX Laboratory Workstation (Beckman Coulter) will then stack the two plates and the Sarstedt plates containing the purified proteins are sealed and frozen. When appropriate, the method “F 1×96 Test Expression (Stop Digest)” on the Biomek FX Laboratory Workstation (Beckman Coulter) is opened. The air is turned on from the blue switch located on the upper right side of the Biomek FX Laboratory Workstation (Beckman Coulter) (the arrow indicates “ON”). The method is started by clicking the green play key on the toolbar. The labware is loaded as depicted by the instrument setup window. “OK” is clicked. The system will indicate that the plates will be ready in 2 min (“OK” is clicked to continue). The Biomek FX Laboratory Workstation (Beckman Coulter) will unstack the trypsin digest plates and dispense 5 μL of 20% acetic acid into each well.

Tryptic fragments are purified as follows. 250 μl of dry bulk C18 reverse phase resin is added to a 1.5 ml tube and washed two times with methanol and two times with 75% acetonitrile/1% acetic acid prior to use. An approximate 5:1 slurry is prepared with 75% acetonitrile/1% acetic acid. 10 μl of C18 slurry is added to the trypsin-digested protein In the trypsin digest plates. The resin should float on top of the liquid, and shaken at moderate speed (500-700 rpm) on orbital shaker for 30 min at room temperature. The supernatant is removed by placing the pipette tip below the surface of the liquid to avoid aspirating any resin. Once liquid is removed 200 μl of 2% acetonitrile/1% acetic acid is added to the resin and shaken briefly for 5-15 min at moderate speed (500-700 rpm) on orbital shaker at room temperature.

A 384 well Melt Blown Polypropylene (MBPP; Whatman, UK) filter plate is prepared by washing the wells that will be used with 100 μl 75% acetonitrile/1% acetic acid. The plate is centrifuged for 1-2 min at 1000 rpm and the wash is repeated once. All of the remaining liquid is removed by a second centrifugation if required.

Elution of the peptides from the resin is accomplished by the addition of 15 μl of 75% acetonitrile/1% acetic acid to the C18 resin in the trypsin digest plates; the resin will mix with the elution buffer to form a slurry which will slowly settle to the bottom of the well. The plate is pulse shaken at high speed on the orbital shaker and incubated approximately 5 min. Essentially all of the resin will have entered into a slurry. The plate is centrifuged for 1-2 min at 1000 rpm. The liquid is transferred by either a multichannel pipette or liquid handling system (some resin will be removed as well) to a MBPP filter plate, fitted with a 384 well collection plate, which is then centrifuged for 3-5 min at 1000 rpm. The samples are removed from the 384 well collection plate and placed in a clean 96 well plate. The plate is covered with mat lid and stored in −70 degree Celsius freezer.

Samples can also be prepared for mass spectrometry with Zip Tip_{C}18 as follows. 100% methanol, 2% acetonitrile/1% acetic acid and 65% acetonitrile/1% acetic acid are dispensed into 3 separate solution basins (Labcor, cat#730-004). Using, e.g., the 12-channel Biohit electronic pipettor, ZipTips (Millipore, cat.# ZTC18S960) are wetted by aspirating and dispensing 100% methanol 5×; followed by 2% acetonitrile/1% acetic acid (5×), followed by 65% acetonitrile/1% acetic (5×); and finally with 2% acetonitrile/1% acetic acid (5×). The digested proteins are bound to ZipTips by aspirating and dispensing the samples 20 times. Salts are removed by washing ZipTips with 2% acetonitrile/1% acetic acid (5×). 10 μL of 65% acetonitrile/1% acetic acid are aspirated and dispensed into Nunc 96-well microtitire plate (cat.# 249946).

The samples are then spotted automatically by a modified Gilson 215 or a Biomek FX Laboratory Workstation (Beckman Coulter) onto a mass spectrometry plate and mass spectrometry is conducted as described previously for the manual method.

The results of the mass spectrometry are interpreted as follows. Samples destined for MS clean-up and MS analysis are tracked using the Sample Tracker, MSQuant Output software packages, as well as freezer and instrument logs.

The set-up requirements are as follows; the Sample Tracker Software is opened and the clone list is inserted into the sample column on the 96-well list sheet. The appropriate suffix is added and along with the date processed. The suffix is of the form _Testx_Y_His, where Y is either 's' for soluble or ‘i’ for insoluble. If necessary additional information can be placed after the “His” portion of the suffix. After deciding upon which suffix is to be added to the names, the box to add the suffix to the clone list is clicked. All other worksheets in the Sample Tracker software will be updated automatically. A copy of the Sample Tracker file is saved as ‘Testx_XX_YYMMDD’, where X is the organism identifier and YYMMDD is the date on which the samples were cleaned up.

Following MS analysis, the peak list is saved as “Testx_XX_YYMMDD” in Data Archiver/BrukerTBA. (Additional information can be added after the YYMMDD, if necessary). Prior to running the Knexus Software ensure only the following parameters are checked: Z % (i.e., the likelihood that the protein found is actually the protein in the well), Protein Information, %, kDa, ®, Data File. The Knexus report is run and saved as “Testx_XX_YYMMDD” in Data Archiver/Knexus YYYY (where YYYY is the current year).

The MSQuant software package is opened and the required information is filled in the Report Info box; MS Date is the date the samples were shot; MSQuant Report date is the date the MSQuant analysis was performed. In the File Locations box, the name of the test expression assay is entered which should be identical to that given to the Knexus report and data peak list. The software's parameters can be modified by clicking the “Constants” bar and entering the appropriate changes in the drop down box. The clone list including clone ID, MW and PCR results must be inserted into the correct area. Once all of the required information has been entered, the analysis is initiated by clicking, “Load Data.” The MS raw data and the Knexus report will be inserted into the similarly named worksheets. A preliminary R&D Quant report will be generated. In the Knexus report worksheet, the spectra that have been positively identified (i.e., Z score >85%) but whose clone name in the Protein Information column does not match that in the Data file column of the Knexus report will be highlighted blue. The spectra of the clones that have not met specified criteria (12 or more peaks and under 85% Z score) are flagged in the “Check me” column of the R&D MSQuant report worksheet.

For clones that produce a “Check me,” the spectra can be opened in M/Z by double-clicking on the clone name in the Data file column. Each spectrum is manually calibrated, labeled, and run through the Profound database search. If a match is found, the Z % value on the Knexus report is changed to 101% and the appropriate clone name is entered into the Protein Information column (i.e. the name of the clone in the Data File should be the same as the clone in Protein Information). All “Check me” clones are manually interpreted in the above manner. Once all the “Check me” clones have been re-evaluated and the changes made in the Knexus Report worksheet, the “Update Report” button is clicked on the General Information worksheet. The modified report will be presented in the worksheet entitled “Cloning Quant Report.” A copy of this file is saved as “Testx_XX_YYMMDD_additional information” (where XX is the organism identifier, YYMMDD is the date on which the samples were prepared and shot in the MALDI, and additional information is any additional information that is added to the report name by the mass spectrometrist).

The following information is extracted from the MSQuant report:

| Item | Description |
|---|---|
| Clone Identification | The name of the protein |
| Plate Position | The location of the protein on the MALDI-ToF |
| | sample plate |
| Expression | A simple Yes/No explaining whether the protein |
| | planted was the protein produced. |
| Z % | The likelihood that the protein found is actually the |
| | protein in the well. |
| Molecular Weight | The molecular weight of the protein |
| % Soluble | The ratio of soluble protein to insoluble protein |
| Soluble Quantity | The expression level of soluble protein per litre |
| (mg/L) | growth media |
| Insoluble Quantity | The expression level of insoluble protein per litre |
| (mg/L) | growth media |
| Growth Conditions | Related to the % Soluble (simply if >X then A, if |
| | >Y then B, etc . . . ) |
| Date | Month, Day, Year |
| MS-ID | The mass spectrometer operator |
| Raw Data Link | Hyperlink to the Data File |

The following data is extracted from the Knexus Report or calculated by the MSQuant report as follows:

| Item | Location |
|---|---|
| Clone Identification | from the Knexus Report data file name |
| Plate Position | Position of the plate |
| Expression | If the Z % is <85 then NO |
| | If the Z % is >=85 then |
| | If the Clone Identification = Protein Found |
| | (extract from Knexus Report) then YES |
| | Else NO |
| Z % | from Knexus Report |
| Molecular Weight | from Knexus Report |
| % Soluble | Calculated in MSQuant |
| Soluble Quantity (mg/L) | Calculated in MSQuant |
| Insoluble Quantity (mg/L) | Calculated in MSQuant |
| Growth Conditions | If soluble quantity >X then it's A else if it's |
| | >Y then it's B etc . . . Where A and B are |
| | different growth conditions. |
| Date | Month, Day, Year |
| MS-ID | mass spectrometer operator |
| Raw Data Link | from the Knexus Report |

The MSQuant and Knexus reports are HTML files. Microsoft Excel can easily import these files and extract the required data. The program could also be done as a Visual Basic application that creates the Excel file or there could be an Excel template with a button that runs visual basic script and fills in the cells.

Equivalents

The present invention provides among other methods for high throughput purification, characterization, and identification of recombinant proteins. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. To the extent that any U.S. Provisional Patent Applications to which this patent application claims priority incorporate by reference another U.S. Provisional Patent Application, such other U.S. Provisional Patent Application is not incorporated by reference herein unless this patent application expressly incorporates by reference, or claims priorty to, such other U.S. Provisional Patent Application.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: WO 03/02724, U.S. Pat. No. 6,291,192, U.S. Pat. No. 6,020,141, U.S. Pat. No. 5,959,738, U.S. Pat. No. 6,268,158, U.S. Pat. No. 6,232,085, WO 00/45168, WO 00/79238, WO 00/77712, EP 1047108, EP 1047107, WO 00/72004, WO 00/73787, WO00/67017, WO 00/48004, WO 01/48209, WO 00/45168, WO 00/45164, U.S. Ser. No. 09/720,272; PCT/CA99/00640; PCT/CA02/01428; U.S. patent application Ser. No. 10/370,268 (filed Feb. 20, 2003); Ser. No. 10/097,125 (filed Mar. 12, 2002); Ser. No. 10/097,193 (filed Mar. 12, 2002); Ser. No. 10/202,442 (filed Jul. 24, 2002); Ser. No. 10/097,194 (filed Mar. 12, 2002); Ser. No. 09/671,817 (filed Sep. 17, 2000); Ser. No. 09/965,654 (filed Sep. 27, 2001); Ser. No. 09/727,812 (filed Nov. 30, 2000); No. 60/370,667 (filed Apr. 8, 2002); a utility patent application entited “Methods and Appartuses for Purification” (filed Sep. 18, 2002); U.S. Pat. Nos. 6,451,591; 6,254,833; 6,232,114; 6,229,603; 6,221,612; 6,214,563; 6,200,762; 6,171,780; 6,143,492; 6,124,128; 6,107,477; D428157; 6063338; 6004808; 5985214; 5981200; 5928888; 5910287; 6248550; 6232114; 6229603; 6221612; 6214563; 6200762; 6197928; 6180411; 6171780; 6150176; 6140132; 6124128; 6107066; 6270988; 6077707; 6066476; 6063338; 6054321; 6054271; 6046925; 6031094; 6008378; 5998204; 5981200; 5955604; 5955453; 5948906; 5932474; 5925558; 5912137; 5910287; 5866548; 6214602; 5834436; 5777079; 5741657; 5693521; 5661035; 5625048; 5602258; 5552555; 5439797; 5374710; 5296703; 5283433; 5141627; 5134232; 5049673; 4806604; 4689432; 4603209; 6217873; 6174530; 6168784; 6271037; 6228654; 6184344; 6046133; 5910437; 5891993; 5854389; 5792664; 6248558; 6341256; 5854922; and 5866343; Brooks et al. (1983) J Comput Chem 4:187-217; Weiner et al (1981) J Comput. Chem. 106: 765; Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488; Ryckaert et al. (1977) J Comput Phys 23:327; Van Gunsteren et al. (1977) Mol Phys 34:1311; Anderson (1983) J Comput Phys 52:24; J. Mol. Biol. 48: 442-453, 1970; Dayhoff et al., Meth. Enzymol. 91: 524-545, 1983; Henikoff and Henikoff, Proc. Nat. Acad. Sci. USA 89: 10915-10919, 1992; J. Mol. Biol. 233: 716-738, 1993; Methods in Enzymology, Volume 276, Macromolecular crystallography, Part A, ISBN 0-12-182177-3 and Volume 277, Macromolecular crystallography, Part B, ISBN 0-12-182178-1, Eds. Charles W. Carter, Jr. and Robert M. Sweet (1997), Academic Press, San Diego; Pfuetzner, et al., J. Biol. Chem. 272: 430-434 (1997); U.S. Pat. Nos. 5,668,734; 6,194,179; 6,162,627; 6,043,024; 5,817,474; 5,891,642; 5,989,827; 5,891,643; 6,077,682; WO 00/05414; WO 99/22019; Cavanagh, et al., Protein NMR Spectroscopy, Principles and Practice, 1996, Academic Press; Clore, et al., NMR of Proteins. In Topics in Molecular and Structural Biology, 1993, S. Neidle, Fuller, W., and Cohen, J. S., eds., Macmillan Press, Ltd., London; and Christendat et al., Nature Structural Biology 7: 903-909 (2000).

1. A method for high throughput determination of the identity, quantity and solubility profile of a plurality of recombinant proteins, comprising:
providing a plurality of lysates, wherein each lysate comprises a recombinant protein linked to a tag peptide and a proteolytic enzyme recognition site located between the recombinant protein and the tag peptide, wherein the tag peptide and the proteolytic enzyme recognition site are the same for each of the recombinant proteins and wherein each lysate is provided in a well of a multi-well plate;
separating the soluble and the insoluble biological material of the lysates, to obtain from each lysate a fraction comprising the insoluble biological material and a fraction comprising the soluble biological material;
subjecting one or both of the fractions comprising the soluble and insoluble biological material separately to tag peptide affinity chromatography in a multi-well plate to obtain affinity purified recombinant proteins from one or both of the fractions of each lysate;
proteolytically digesting the affinity purified recombinant proteins from one or both of the fractions with a proteolytic enzyme in the presence of an internal quantification standard in a multi-well plate, wherein the proteolytic enzyme cleaves the proteolytic enzyme recognition site and wherein the internal quantification standard consists essentially of a chemically modified form of the tag peptide;
subjecting the proteolytic fragments to MALDI-TOF, ion trap or electrospray mass spectrometry in a multi-well plate to obtain a mass spectrum; and
determining the quantity of the plurality of recombinant proteins in one or both of the soluble and insoluble fractions, by comparing the intensity of the peak of the tag peptide in the mass spectrum of the soluble or insoluble fraction to that of the internal quantification standard in the mass spectrum of the soluble or insoluble fraction, respectively, to thereby determine the identity, solubility profile, and quantity of the recombinant protein.

2. The method of claim 1, wherein determining the solubility profile and quantity of the plurality of recombinant proteins is conducted using software.

3. The method of claim 2, wherein the software is the MS Quant software.

4. The method of claim 1, further comprising determining the identity of the plurality of proteins, comprising comparing the mass spectrum with that of proteins in a database.

5. The method of claim 4, comprising using software to compare the mass spectrum with that of proteins in a database.

6. The method of claim 5, comprising using MS Quant software.

7. The method of claim 1, wherein each lysate is a lysate of a clone of host cells, wherein each clone comprises a recombinant protein linked to a tag peptide and a proteolytic enzyme recognition site located between the recombinant protein and the tag peptide.

8. The method of claim 7, comprising
first providing a plurality of clones of host cells, wherein each clone is provided in a well of a multi-well plate; and
lysing the plurality of clones of host cells in the multi-well plate to obtain a plurality of lysates.

9. The method of claim 8, wherein the host cells are prokaryotic host cells.

10. The method of claim 9, wherein the host cells are eukaryotic host cells.

11. The method of claim 1, wherein each lysate derives from an in vitro transcription and translation lysate.

12. The method of claim 11, further comprising:
providing a plurality of RNAs encoding the plurality of recombinant proteins, wherein each RNA is provided in a well of a multi-well plate; and
in vitro translating the RNAs to produce a plurality of lysates, wherein each lysate comprises a recombinant protein.

13. The method of claim 12, further comprising:
providing a plurality of nucleic acids encoding the plurality of recombinant proteins, wherein each nucleic acid is provided in a well of a multi-well plate; and
in vitro transcribing the nucleic acids to produce the plurality of RNAs encoding the plurality of recombinant proteins.

14. The method of claim 13, further comprising amplifying the plurality of nucleic acids in the multi-well plate to obtain amplified nucleic acids prior to in vitro transcribing the nucleic acids.

15. The method of claim 14, further comprising isolating the amplified nucleic acids prior to in vitro transcribing the nucleic acids.

16. The method of claim 1, wherein the multi-well plate is a 96-well plate.

17. The method of claim 1, wherein the multi-well plate is a 384-well plate.

18. The method of claim 1, wherein the plurality of recombinant proteins is at least 10 recombinant proteins.

19. The method of claim 18, wherein the plurality of recombinant proteins is at least 100 recombinant proteins.

20. The method of claim 19, wherein the plurality of recombinant proteins is at least 1000 recombinant proteins and the lysates are in a plurality of multi-well plates.

21. The method of claim 1, wherein the affinity chromatography is a chromatography step using a resin selected from the group consisting of a metal ion resin; glutathione-S-transferase (GST) resin; maltose resin; lectin resin; or a resin coupled to a ligand of the tag peptide.

22. The method of claim 21, wherein the affinity resin is a Ni⁺⁺ resin and the tag peptide contains polyhistidine.

23. The method of claim 1, wherein the proteolytic enzyme is trypsin.

24. The method of claim 1, wherein the internal quantification standard is an isotopically labeled form of the tag peptide.

25. The method of claim 24, wherein the internal quantification standard is ¹⁵N labeled peptide containing polyhistidine.

26. The method of claim 1, further comprising purifying the proteolytic fragments prior to mass spectrometry.

27. The method of claim 26, wherein the proteolytic fragments are purified by chromatography over C18 reverse phase resin.

28. The method of claim 1, further comprising removing an aliquot of the affinity purified recombinant proteins from one or both of the fractions prior to proteolytically digesting the affinity purified recombinant proteins.

29. The method of claim 28, which further comprises subjecting the undigested aliquot of the affinity purified recombinant proteins to structural or biochemical analysis.

30. The method of claim 29, wherein the structural or biochemical analysis is an activity assay.

31. The method of claim 29, wherein the structural or biochemical analysis is a binding assay.

32. The method of claim 31, wherein the binding assay is used to identify or characterize the interaction between the affinity purified recombinant proteins and one or more of a polypeptide, a polynucleotide, or a small molecule.

33. The method of claim 29, wherein the structural or biochemical analysis is an assay to determine the specific activity of the protein.

34. The method of claim 29, wherein the structural or biochemical analysis is characterization of the structure of the protein using one or more of NMR, x-ray crystallography, and mass spectroscopy.

35. The method of claim 29, wherein the structural or biochemical analysis is a crystallization screen to determine conditions suitable for crystallization of the affinity purified recombinant protein.

36. The method of claim 1, wherein the plurality of recombinant proteins are comprised in the fraction comprising the insoluble biological material of each lysate.

37. The method of claim 36, wherein the recombinant proteins comprised in the fraction comprising the insoluble biological material of each lysate are membrane associated proteins.

38. The method of claim 1, wherein the plurality of lysates are obtained from a plurality of clones of host cells.

39. The method of claim 38, wherein the plurality of clones of host cells are grown under the same conditions prior to lysis.

40. The method of claim 38, wherein the plurality of clones of host cells comprise two or more nucleic acids encoding for related polypeptides.

41. The method of claim 40, wherein the two or more nucleic acids encode polypeptides that differ from each other by the addition, substitution, or deletion of at least one amino acid residue.

42. The method of claim 41, wherein a plurality of nucleic acids encode a plurality of related polypeptides.

43. The method of claim 1, wherein the plurality of lysates are obtained from at least one host cell clone grown under a variety of growth conditions.

44. The method of claim 43, wherein the growth conditions are one or more of the following:
time, temperature, culture media, and presence of a label.

45. The method of claim 43, which further comprises comparing one or more of the identity, solubility profile, and quantity of the recombinant protein obtained from the plurality of lysates thereby evaluating the growth conditions for affects on one or both of protein expression and solubility.

46. The method of claim 45, which further comprises determining the optimal growth conditions for one or both of protein expression and solubility.

47. The method of claim 1, wherein the plurality of lysates are obtained from at least one host cell clone grown in the presence of a label under a variety of growth conditions.

48. The method of claim 47, which further comprises determining the amount of label incorporated into the recombinant protein in each of the plurality of lysates and comparing one or more of the amount of label incorporated, percent of recombinant proteins labeled, solubility profile, and quantity of the recombinant protein obtained from the plurality of lysates thereby evaluating the growth conditions for affects on one or more of protein expression, solubility, and efficiency of labeling.

49. The method of claim 48, wherein determining the amount of label incorporated into the recombinant protein is determined using mass spectrometry.

50. The method of claim 1, wherein affinity purification of the recombinant proteins from one or both of the soluble and insoluble fractions from each lysate produces at least 1 μg of protein from each lysate.

51. A method for high throughput determination of the solubility profile and quantity of a plurality of recombinant proteins, comprising:
providing a plurality of clones of host cells, wherein each clone comprises a recombinant protein linked to a tag and a proteolytic enzyme recognition site located between the recombinant protein and the tag peptide, wherein the tag and the proteolytic enzyme recognition site are the same for each of the recombinant proteins and wherein each clone is provided in a well of a multi-well plate;
lysing the plurality of clones of host cells in the multi-well plate to obtain first lysates;
subjecting the first lysates to centrifugation in a multi-well plate to collect insoluble material in pellets and soluble material in first supernatants;
transferring the first supernatants to wells of a multi-well plate;
adding denaturing buffer to the pellets in the multi-well plate to obtain second lysates;
subjecting the second lysates to centrifugation to collect denatured insoluble material in pellets and denatured soluble material in second supernatants;
subjecting one or both of the first and second supernatants separately to tag peptide affinity chromatography in a multi-well plate to obtain one or both of affinity purified soluble protein fractions and affinity purified denatured soluble recombinant protein fractions;
proteolytically digesting the affinity purified recombinant proteins with a proteolytic enzyme in the presence of an internal quantification standard in a multi-well plate to obtain proteolytic fragments of recombinant proteins, wherein the proteolytic enzyme cleaves the proteolytic enzyme recognition site and wherein the internal quantification standard consists essentially of a chemically modified form of the tag peptide;
purifying the proteolytic fragments in a multi-well plate to obtain purified proteolytic fragments;
subjecting the purified proteolytic fragments to MALDI-TOF, ion trap or electrospray mass spectrometry in a multi-well plate; and
determining the quantity of the plurality of recombinant proteins in one or both of the soluble and denatured soluble recombinant protein fractions, by comparing the intensity of the peak of the tag peptide in the mass spectrum of the soluble or denatured soluble recombinant protein fractions to that of the internal quantification standard in the mass spectrum of the soluble or denatured soluble recombinant protein fractions, respectively, to thereby determine the solubility profile and quantity of the recombinant protein.

52. A method for high throughput determination of the quantity of a plurality of recombinant proteins, comprising:
providing a plurality of purified recombinant proteins, wherein each recombinant protein comprises a tag peptide and a proteolytic enzyme recognition site located between the recombinant protein and the tag peptide, wherein the tag peptide and the proteolytic enzyme recognition site are the same for each of the recombinant proteins and wherein each recombinant protein is provided in a well of a multi-well plate;
proteolytically digesting the recombinant proteins with a proteolytic enzyme in the presence of an internal quantification standard in a multi-well plate, wherein the proteolytic enzyme cleaves the proteolytic enzyme recognition site and wherein the internal quantification standard consists essentially of a chemically modified form of the tag peptide;
subjecting the proteolytic fragments to MALDI-TOF, ion trap or electrospray mass spectrometry in a multi-well plate to obtain a mass spectrum; and
determining the quantity of the plurality of recombinant proteins, by comparing the intensity of the peak of the tag peptide in the mass spectrum to that of the internal quantification standard in the mass spectrum, to thereby determine the quantity of the recombinant protein.

53. A kit for high throughput purification, determination of the solubility profile and quantification of a plurality of recombinant proteins, comprising a vector for expressing recombinant proteins in host cells; affinity chromatography resin; a proteolytic enzyme; an internal quantification standard; a matrix for MALDI-TOF mass spectrometry; and instructions for use.

54. The kit of claim 53, further comprising at least one buffer selected from the group consisting of a lysis buffer; a denaturing buffer; an affinity chromatography binding buffer; an affinity chromatography washing buffer; an affinity chromatography elution buffer; and a proteolytic digestion buffer.

55. The kit of claim 53, further comprising at least one multi-well plate.

56. A computer for determining the amount of a plurality of proteins; identifying a plurality of proteins; and/or determining the solubility profile of a plurality of proteins, comprising:
(a) a machine-readable data storage medium comprising a data storage material encoded with machine-readable data, wherein said data comprises data obtained from MS analysis of a plurality of recombinant proteins according to the method of claim 1;
(b) a working memory for storing instructions for processing said machine-readable data of (a);
(c) a central-processing unit coupled to said working memory and to said machine-readable data storage medium for extracting information from the data on the machine-readable storage medium; and
(d) a display coupled to said central-processing unit for displaying said results.

57. A business method for providing the amount of a plurality of proteins; identifying a plurality of proteins; and/or determining the solubility profile of a plurality of proteins, comprising:
(a) receiving MS results obtained essentially according to the method of claim 1 from a sender via a network;
(b) analyzing the MS results of (a) according to the method of claim 1 to obtain the amount of a plurality of proteins; identifying a plurality of proteins; and/or determining the solubility profile of a plurality of proteins; and
(c) sending at least part of the results to the sender via a network.

58. A plurality of compositions comprising a plurality of recombinant proteins wherein the identity, quantity and solubility profile of the recombinant proteins is determined, and wherein the plurality of recombinant proteins were purified using a method comprising:
providing a plurality of lysates, wherein each lysate comprises a recombinant protein linked to a tag peptide and a proteolytic enzyme recognition site located between the recombinant protein and the tag peptide, wherein the tag peptide and the proteolytic enzyme recognition site are the same for each of the recombinant proteins and wherein each lysate is provided in a well of a multi-well plate;
separating the soluble and the insoluble biological material of the lysates, to obtain from each lysate a fraction comprising the insoluble biological material and a fraction comprising the soluble biological material;
subjecting one or both of the fractions comprising the soluble and insoluble biological material separately to tag peptide affinity chromatography in a multi-well plate to obtain affinity purified recombinant proteins from one or both of the fractions of each lysate;
proteolytically digesting the affinity purified recombinant proteins from one or both of the fractions with a proteolytic enzyme in the presence of an internal quantification standard in a multi-well plate, wherein the proteolytic enzyme cleaves the proteolytic enzyme recognition site and wherein the internal quantification standard consists essentially of a chemically modified form of the tag peptide;
subjecting the proteolytic fragments to MALDI-TOF, ion trap or electrospray mass spectrometry in a multi-well plate to obtain a mass spectrum; and
determining the quantity of the plurality of recombinant proteins in one or both of the soluble and insoluble fractions, by comparing the intensity of the peak of the tag peptide in the mass spectrum of the soluble or insoluble fraction to that of the internal quantification standard in the mass spectrum of the soluble or insoluble fraction, respectively, to thereby determine the identity, solubility profile, and quantity of the recombinant protein.
